(19) United States
(12) Patent Application Publication
(10) Pub. No.: US 2008/0214503 A1
(43) Pub. Date: Sep. 4, 2008

(54) Hiv Integrase Inhibitors

(75) Inventors: Brian A. Johns, Durham, NC (US); Andrew Spaltenstein, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SmithKline Beecham Corporation

(21) Appl. No.: 11/997,780

(22) PCT Filed: Jul. 28, 2006
(86) PCT No.: PCT/US2006/029652

§ 371 Date: Feb. 4, 2008

Related U.S. Application Data
(60) Provisional application No. 60/705,328, filed on Aug. 4, 2005.

(51) Int. Cl. A61K 31/675; C07D 471/04; A61K 31/4375; A61P 31/12; C07F 9/58

(52) U.S. Cl. 514/81; 546/123; 514/300; 546/23

(57) ABSTRACT

The present intention features compounds that are HIV integrase inhibitors and may be useful in the inhibition of HIV replication, the prevention and/or treatment of infection by HIV, and in the treatment of AIDS and/or ARC.

### BACKGROUND OF THE INVENTION

The human immunodeficiency virus (“HIV”) is the causative agent for acquired immunodeficiency syndrome (“AIDS”), a disease characterized by the destruction of the immune system, particularly of CD4⁺T-cells, with attendant susceptibility to opportunistic infections, and its precursor AIDS-related complex (“ARC”), a syndrome characterized by symptoms such as persistent generalized lymphadenopathy, fever and weight loss. HIV is a retrovirus; the conversion of its RNA to DNA is accomplished through the action of the enzyme reverse transcriptase. Compounds that inhibit the function of reverse transcriptase inhibit replication of HIV in infected cells. Such compounds are useful in the prevention or treatment of HIV infection in humans.

A required step in HIV replication in human T-cells is the insertion by virally-encoded integrase of proviral DNA into the host cell genome. Integration is believed to be mediated by integrase in a process involving assembly of a stable nucleoprotein complex with viral DNA sequences, cleavage of two nucleotides from the 3′ termini of the linear proviral DNA and covalent joining of the recessed 3′ OH termini of the proviral DNA at a staggered cut made at the host target site. The repair synthesis of the resultant gap may be accomplished by cellular enzymes.

There is continued need to find new therapeutic agents to treat human diseases. HIV integrase is an attractive target for the discovery of new therapeutics due to its important role in viral infections, particularly HIV infections.

### SUMMARY OF THE INVENTION

The present invention features compounds that are HIV integrase inhibitors and therefore are useful in the inhibition of HIV replication, the prevention and/or treatment of infection by HIV, and in the treatment of AIDS and/or ARC. The present invention features compounds of formula (I):

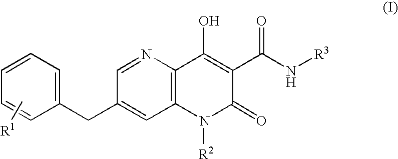

wherein:

R¹ is one or more substituents independently selected from hydrogen, hydroxy, CN, N(R^{a}R^{b}), C₁₋₈alkyl, C₃₋₇ cycloalkyl, halogen and C₁₋₈ alkoxy;

R² is selected from hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})N(R^{a}), C(SR^{a})═N(R^{b}), C(OR^{a})═N(R^{b}), N(R^{a})C(SR^{a})═N(R^{b}) and heterocycle optionally substituted with oxo or R^{a};

or optionally when R² is C₅₋₇ cycloalkyl, C₆₋₁₄ aralkyl, C₅₋₇ cycloalkenyl, C₆₋₁₄ aryl or heterocycle R² may be fused to 5-7 membered carbocyclic or heterocyclic rings;

R³ is

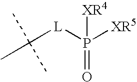

wherein

L is C₁₋₈alkyl optionally substituted with C(O)NHR⁷;

X is O or NHR⁶;

R⁴ and R⁵ are independently hydrogen; C₁₋₈alkyl optionally substituted with C(O)R^{a} or C(O)NR^{a}R^{b}; or C₆₋₁₄aryl optionally substituted with halogen, alkoxy or NR^{a}R^{b};

R⁶ is hydrogen, C₁₋₈alkyl optionally substituted with OR⁷, or C₆₋₁₄aryl;

R⁷ is hydrogen or C₁₋₈alkyl;

R^{a} and R^{b} are independently hydrogen, NO₂, OR^{c}, CN, N(R^{c}R^{d}), C(O)R^{c}, C(O)C(O)R^{c}, C(O)N(R^{c}R^{d}), C(O)C(O)N(R^{c}R^{d}), S(O)ₘR^{c}, SR^{c}, S(O)ₘN(R^{c}R^{d}), C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, CN, NO₂, OR^{c}, N(R^{c}R^{d}), S(O)ₘR^{c}, SR^{c}, OS(O)ₘR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}), N(R^{c})S(O)ₘOR^{d}, C(O)R^{c}, OC(O)R^{c}, C(O)OR^{c}, OC(O)OR^{c}, N(R^{c})C(O)R^{d}, C(O)N(R^{c}R^{d}), N(R^{c})C(O)N(R^{c}R^{d}), OC(O)N(R^{c}R^{d}), N(R^{c})C(O)OR^{d}, C(NR^{c}R^{d})═N(R^{c}), C(SR^{c})═N(R^{d}), C(OR^{c})N(R^{d}) and heterocycle;

Optionally, R^{a} and R^{b} may be linked together through one or more ring carbon atoms and/or ring heteroatoms including N, O, C(R^{c}R^{d}), C(O), S(O)ₘ, or S to form a saturated or unsaturated 3 to 8 membered carbocyclic or heterocyclic ring;

R^{c}C and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;

Optionally, R^{c} and R^{d} may be linked together through one or more ring carbon atoms and/or ring heteroatoms including N, O, C(O) and S(O)ₘ, or S to form a saturated or unsaturated 3 to 8 membered carbocyclic or heterocyclic ring;

m is 1 or 2;

or a pharmaceutically acceptable salt thereof.

### DETAILED DESCRIPTION OF THE INVENTION

The present invention includes the compounds of Formula (I), useful in treating or preventing viral infections, particularly HIV infections, pharmaceutical compositions comprising compounds of Formula (I), and processes for preparing the compounds.

The present invention features compounds of formula (I):

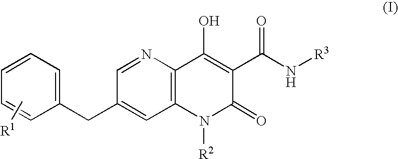

wherein:

R¹ is one or more substituents independently selected from hydrogen, hydroxy, CN, N(R^{a}R^{b}), C₁₋₈alkyl, C₃₋₇ cycloalkyl, halogen and C₁₋₈ alkoxy;

R² is selected from hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a};

or optionally when R² is C₅₋₇ cycloalkyl, C₆₋₁₄ aralkyl, C₅₋₇ cycloalkenyl, C₆₋₁₄ aryl or heterocycle R² may be fused to 5-7 membered carbocyclic or heterocyclic rings;

R³ is

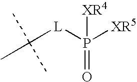

wherein:

L is C₁₋₈alkyl optionally substituted with C(O)NHR⁷;

X is O or NHR⁶;

R⁴ and R⁵ are independently hydrogen; C₁₋₈alkyl optionally substituted with C(O)R^{a} or C(O)NR^{a}R^{b}; or C₆₋₁₄aryl optionally substituted with halogen, alkoxy or NR^{a}R^{b};

R⁶ is hydrogen, C₁₋₈alkyl optionally substituted with OR⁷, or C₆₋₁₄aryl;

R⁷ is hydrogen or C₁₋₈alkyl;

R^{a} and R^{b} are independently hydrogen, NO₂, OR^{c}, CN, N(R^{c}R^{d}), C(O)R^{c}, C(O)C(O)R^{c}, C(O)N(R^{c}R^{d}), C(O)C(O)N(R^{c}R^{d}), S(O)ₘR^{c}, SR^{c}, S(O)ₘN(R^{c}R^{d}), C₁₋₈alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, CN, NO₂, OR^{c}, N(R^{c}R^{d}), S(O)ₘR^{c}, SR^{c}, OS(O)ₘR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}), N(R^{c})S(O)ₘOR^{d}, C(O)R^{c}, OC(O)R^{c}, C(O)OR^{c}, OC(O)OR^{c}, N(R^{c})C(O)R^{d}, C(O)N(R^{c}R^{d}), N(R^{c})C(O)N(R^{c}R^{d}), OC(O)N(R^{c}R^{d}), N(R^{c})C(O)OR^{d}, C(NR^{c}R^{d})═N(R^{c}), C(SR^{c})═N(R^{d}), C(OR^{c})═N(R^{d}) and heterocycle;

Optionally, R^{a} and R^{b} may be linked together through one or more ring carbon atoms and/or ring heteroatoms including N, O, C(R^{c}R^{d}), C(O), S(O)ₘ, or S to form a saturated or unsaturated 3 to 8 membered carbocyclic or heterocyclic ring;

R^{c}C and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;

Optionally, R^{c}C and R^{d} may be linked together through one or more ring carbon atoms and/or ring heteroatoms including N, O, C(O) and S(O)ₘ, or S to form a saturated or unsaturated 3 to 8 membered carbocyclic or heterocyclic ring;

m is 1 or 2;

or a pharmaceutically acceptable salt thereof.

The term “alkyl”, alone or in combination with any other term, refers to a straight-chain or branched-chain saturated aliphatic hydrocarbon radical containing the specified number of carbon atoms. Examples of alkyl radicals include, but are not limited to, methyl, ethyl, n-propyl, isopropyl, n-butyl, isobutyl, sec-butyl, tert-butyl, pentyl, isoamyl, n-hexyl and the like.

The term “cycloalkyl” refers to a saturated or partially saturated carbocyclic ring composed of 3-6 carbons in any chemically stable configuration. Examples of suitable carbocyclic groups include cyclopropyl, cyclobutyl, cyclopentyl, cyclohexyl, and cyclohexenyl.

The term “alkenyl,” alone or in combination with any other term, refers to a straight-chain or branched-chain alkyl group with at least one carbon-carbon double bond. Examples of alkenyl radicals include, but are not limited to, ethenyl, propenyl, isopropenyl, butenyl, isobutenyl, pentenyl, hexenyl, hexadienyl and the like.

The term “alkynyl” refers to hydrocarbon groups of either a straight or branched configuration with one or more carbon-carbon triple bonds which may occur in any stable point along the chain, such as ethynyl, propynyl, butynyl, pentynyl, and the like.

The term “alkoxy” refers to an alkyl ether radical, wherein the term “alkyl” is defined above. Examples of suitable alkyl ether radicals include, but are not limited to, methoxy, ethoxy, n-propoxy, isopropoxy, n-butoxy, isobutoxy, sec-butoxy, tert-butoxy and the like.

The term “aryl” alone or in combination with any other term, refers to a carbocyclic aromatic moiety (such as phenyl or naphthyl) containing the specified number of carbon atoms, preferably from 6-14 carbon atoms, and more preferably from 6-10 carbon atoms. Examples of aryl radicals include, but are not limited to, phenyl, naphthyl, indenyl, azulenyl, fluorenyl, anthracenyl, phenanthrenyl, tetrahydronaphthyl, indanyl, phenanthridinyl and the like. Unless otherwise indicated, the term “aryl” also includes each possible positional isomer of an aromatic hydrocarbon radical, such as in 1-naphthyl, 2-naphthyl, 5-tetrahydronaphthyl, 6-tetrahydronaphthyl, 1-phenanthridinyl, 2-phenanthridinyl, 3-phenanthridinyl, 4-phenanthridinyl, 7-phenanthridinyl, 8-phenanthridinyl, 9-phenanthridinyl and 10-phenanthridinyl. Examples of aryl radicals include, but are not limited to, phenyl, naphthyl, indenyl, azulenyl, fluorenyl, anthracenyl, phenanthrenyl, tetrahydronaphthyl, indanyl, phenanthridinyl and the like.

The term “aralkyl” refers to an alkyl group substituted by an aryl group. Examples of aralkyl groups include, but are not limited to, benzyl, phenethyl and the like.

The term “heterocycle,” “heterocyclic,” and “heterocyclyl” as used herein, refer to a 3- to 7-membered monocyclic heterocyclic ring or 8- to 11-membered bicyclic heterocyclic ring system any ring of which is either saturated, partially saturated or unsaturated, and which may be optionally benzofused if monocyclic. Each heterocycle consists of one or more carbon atoms and from one to four heteroatoms selected from the group consisting of N, O and S, and wherein the nitrogen and sulfur heteroatoms may optionally be oxidized, and the nitrogen atom may optionally be quaternized, and including any bicyclic group in which any of the above-defined heterocyclic rings is fused to a benzene ring. The heterocyclic ring may be attached at any carbon or heteroatom, provided that the attachment results in the creation of a stable structure. Preferred heterocycles include 5-7 membered monocyclic heterocycles and 8-10 membered bicyclic heterocycles. When the heterocyclic ring has substituents, it is understood that the substituents may be attached to any atom in the ring, whether a heteroatom or a carbon atom, provided that a stable chemical structure results. “Heteroaromatics” or “heteroaryl” are included within the heterocycles as defined above and generally refers to a heterocycle in which the ring system is an aromatic monocyclic or polycyclic ring radical containing five to twenty carbon atoms, preferably five to ten carbon atoms, in which one or more ring carbons, preferably one to four, are each replaced by a heteroatom such as N, O, S and P. Preferred heteroaryl groups include 5-6 membered monocyclic heteroaryls and 8-10 membered bicyclic heteroaryls. Also included within the scope of the term “heterocycle, “heterocyclic” or “heterocyclyl” is a group in which a non-aromatic heteroatom-containing ring is fused to one or more aromatic rings, such as in an indolinyl, chromanyl, phenanthridinyl or tetrahydro-quinolinyl, where the radical or point of attachment is on the non-aromatic heteroatom-containing ring. Unless otherwise indicated, the term “heterocycle, “heterocyclic” or “heterocyclyl” also included each possible positional isomer of a heterocyclic radical, such as in 1-indolinyl, 2-indolinyl, 3-indolinyl. Examples of heterocycles include imidazolyl, imidazolinoyl, imidazolidinyl, quinolyl, isoquinolyl, indolyl, indazolyl, indazolinolyl, perhydropyridazyl, pyridazyl, pyridyl, pyrrolyl, pyrrolinyl, pyrrolidinyl, pyrazolyl, pyrazinyl, quinoxolyl, piperidinyl, pyranyl, pyrazolinyl, piperazinyl, pyrimidinyl, pyridazinyl, morpholinyl, thiamorpholinyl, furyl, thienyl, triazolyl, thiazolyl, carbolinyl, tetrazolyl, thiazolidinyl, benzofuranoyl, thiamorpholinyl sulfone, oxazolyl, oxadiazolyl, benzoxazolyl, oxopiperidinyl, oxopyrrolidinyl, oxoazepinyl, azepinyl, isoxozolyl, isothiazolyl, furazanyl, tetrahydropyranyl, tetrahydrofuranyl, thiazolyl, thiadiazoyl, dioxolyl, dioxinyl, oxathiolyl, benzodioxolyl, dithiolyl, thiophenyl, tetrahydrothiophenyl, sulfolanyl, dioxanyl, dioxolanyl, tetahydrofurodihydrofuranyl, tetrahydropyranodihydrofuranyl, dihydropyranyl, tetradyrofurofuranyl and tetrahydropyranofuranyl.

The term “heteroatom” means nitrogen, oxygen, or sulfur and includes any oxidized form of nitrogen, such as N(O) {N⁺—O⁻} and sulfur such as S(O) and S(O)₂, and the quaternized form of any basic nitrogen.

A combination of substituents or variables is permissible only if such a combination results in a stable or chemically feasible compound. A stable compound or chemically feasible compound is one in which the chemical structure is not substantially altered when kept at a temperature of 40° C. or less, in the absence of moisture or other chemically reactive conditions, for at least a week.

Unless otherwise stated, structures depicted herein are also meant to include all stereochemical forms of the structure, i.e., the R and S configurations for each asymmetric center. Therefore, racemates and racemic mixtures, single enantiomers, diastereomeric mixtures and individual diastereoisomers of the present compounds are expressly included within the scope of the invention. Although the specific compounds exemplified herein may be depicted in a particular stereochemical configuration, compounds having either the opposite stereochemistry at any given chiral center or mixtures thereof are also envisioned.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C— or ¹⁴C-enriched carbon are also within the scope of this invention.

It will be apparent to one skilled in the art that certain compounds of this invention may exist in alternative tautomeric forms. All such tautomeric forms of the present compounds are within the scope of the invention. Unless otherwise indicated, the representation of either tautomer is meant to include the other.

The term “pharmaceutically effective amount” refers to an amount effective in treating a virus infection, for example an HIV infection, in a patient either as monotherapy or in combination with other agents. The term “treating” as used herein refers to the alleviation of symptoms of a particular disorder in a patient, or the improvement of an ascertainable measurement associated with a particular disorder, and may include the suppression of symptom recurrence in an asymptomatic patient such as a patient in whom a viral infection has become latent. The term “prophylactically effective amount” refers to an amount effective in preventing a virus infection, for example an HIV infection, or preventing the occurrence of symptoms of such an infection, in a patient. As used herein, the term “patient” refers to a mammal, including a human.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the antiviral agent.

The term “treatment” as used herein refers to the alleviation of symptoms of a particular disorder in a patient, or the improvement of an ascertainable measurement associated with a particular disorder, and may include the suppression of symptom recurrence in an asymptomatic patient such as a patient in whom a viral infection has become latent. Treatment includes prophylaxis which refers to preventing a disease or condition or preventing the occurrence of symptoms of such a disease or condition, in a patient. As used herein, the term “patient” refers to a mammal, including a human.

As used herein, the term “subject” refers to a patient, animal or a biological sample. The term “biological sample”, as used herein, includes, without limitation, cell cultures or extracts thereof; preparations of an enzyme suitable for in vitro assay; biopsied material obtained from a mammal or extracts thereof; and blood, saliva, urine, feces, semen, tears, or other body fluids or extracts thereof.

Throughout this specification, the word “comprise” or variations such as “comprises” or “comprising” will be understood to imply the inclusion of a stated integer or groups of integers but not the exclusion of any other integer or group of integers.

As used herein, the compounds according to the invention are defined to include pharmaceutically acceptable derivatives thereof. A “pharmaceutically acceptable derivative” means any pharmaceutically acceptable salt, ester, salt of an ester, ether, or other derivative of a compound of this invention which, upon administration to a recipient, is capable of providing directly or indirectly a compound of this invention or an inhibitorily active metabolite or residue thereof. Particularly favored derivatives and prodrugs are those that increase the bioavailability of the compounds of this invention when such compounds are administered to a mammal, for example, by allowing an orally administered compound to be more readily absorbed into the blood, or which enhance delivery of the parent compound to a biological compartment, for example, the brain or lymphatic system, relative to the parent species.

The present invention also features a compound of formula (I) wherein:

R¹ is one or more substituents independently selected from hydrogen or halogen;

R is

(a) hydrogen;

(b) C₁₋₈alkyl optionally substituted with C₃₋₇cycloalkyl, OR^{a}, N(R^{a}R^{b}), C(O)R^{a}, C(O)N(R^{a}R^{b}), or heterocycle optionally substituted with oxo or R^{a}; or

(c) C₆₋₁₄aryl optionally substituted with halogen;

R³ is

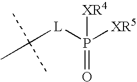

wherein

L is C₁₋₈alkyl optionally substituted with C(O)NHR⁷;

X is O or NHR⁶;

R⁴ and R⁵ are independently hydrogen; C₁₋₈alkyl optionally substituted with C(O)R^{a} or C(O)NR^{a}R^{b}; or C₆₋₁₄aryl optionally substituted with halogen, alkoxy or NR^{a}R^{b};

R⁶ is hydrogen, C₁₋₈alkyl optionally substituted with OR⁷, or C₆₋₁₄aryl;

R⁷ is hydrogen or C₁₋₈alkyl;

wherein R^{a} and R^{b} are independently hydrogen, OR^{c}, SR^{c}, C₁₋₈alkyl, C₆₋₁₄aryl or heterocycle, each of which each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, CN, NO₂, OR^{c}, N(R^{c}R^{d}), S(O)ₘR^{c}, SR^{c}, OS(O)ₘOR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}), N(R^{c})S(O)ₘOR^{d}, C(O)R^{c}, OC(O)R^{c}, C(O)OR^{c}, OC(O)OR^{c}, N(R^{c})C(O)R^{d}, C(O)N(R^{c}R^{d}), N(R^{c})C(O)N(R^{c}R^{d}), OC(O)N(R^{c}R^{d}), N(R^{c})C(O)OR^{d}, C(NR^{c}R^{d})═N(R^{c}), C(SR^{c})═N(R^{d}), C(OR^{c})═N(R^{d}) and heterocycle;

wherein R^{c} is hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;

R^{c} and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;

or a pharmaceutically acceptable salt thereof.

The present invention also features a compound of formula (I) wherein

R¹ is one or more substituents independently selected from hydrogen or halogen;

R² is

(a) hydrogen;

(b) C₁₋₈alkyl optionally substituted with C₃₋₇cycloalkyl, OR^{a}, N(R^{a}R^{b}), C(O)R^{a}, C(O)N(R^{a}R^{b}), or heterocycle optionally substituted with oxo or R^{a}; or

(c) C₆₋₁₄aryl optionally substituted with halogen;

R³ is

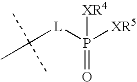

wherein

L is C₁₋₈alkyl optionally substituted with C(O)NHR⁷;

X is O or NHR⁶;

R⁴ and R⁵ are independently hydrogen; C₁₋₈ alkyl optionally substituted with C(O)R^{a} or C(O)NR^{a}R^{b}; or C₆₋₁₄aryl optionally substituted with halogen, alkoxy or NR^{a}R^{b};

R⁶ is hydrogen, C₁₋₈alkyl optionally substituted with OR⁷, or C₆₋₁₄aryl;

R⁷ is hydrogen or C₁₋₈alkyl;

wherein R^{a} and R^{b} are independently hydrogen, NO₂, OR^{c}, C(O)R^{c}, C₁₋₈alkyl optionally substituted with OR^{c}, C(O)OR^{c}, C₆₋₁₄aryl or heterocycle;

wherein R^{c} is hydrogen, C₁₋₈ alkyl or C₆₋₁₄aryl;

or a pharmaceutically acceptable salt thereof.

The present invention further features a compound of formula (I) wherein

R¹ is one or more substituents independently selected from hydrogen or halogen;

R² is C₁₋₈alkyl optionally substituted with C₃₋₇cycloalkyl, OR^{a}, N(R^{a}R^{b}), C(O)R^{a}, C(O)N(R^{a}R^{b}), or heterocycle optionally substituted with oxo; or C₆₋₁₄aryl optionally substituted with halogen;

R³ is

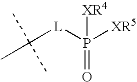

wherein

L is C₁₋₈alkyl optionally substituted with C(O)NHR⁷;

X is O or NHR⁶;

R⁴ and R⁵ are independently hydrogen; C₁₋₈alkyl optionally substituted with C(O)R^{a} or C(O)NR^{a}R^{b}; or C₆₋₁₄aryl optionally substituted with halogen, alkoxy or NR^{a}R^{b};

R⁶ is hydrogen, C₁₋₈alkyl optionally substituted with OR⁷, or C₆₋₁₄aryl;

R⁷ is hydrogen or C₁₋₈alkyl;

wherein R^{a} and R^{b} are independently hydrogen, NO₂, OR^{c}, C(O)R^{c}, C₁₋₈alkyl optionally substituted with OR^{c}, C(O)OR^{c}, C₆₋₁₄aryl or heterocycle;

wherein R^{c} is hydrogen, C₁₋₈ alkyl or C₆₋₁₄aryl;

or a pharmaceutically acceptable salt thereof.

The present invention further features a compound of formula (I) as defined above wherein L is C₁₋₈ alkyl.

The present invention further features a compound of formula (I) as defined above wherein L is C₁₋₈ alkyl and X is O or a pharmaceutically acceptable salt thereof.

The present invention features a compound selected from the group consisting of:
Diethyl {2-[({7-[(4-fluorophenyl)methyl]-4-hydroxy-2-oxo-1,2-dihydro-1,5-naphthyridin-3-yl}carbonyl)amino]ethyl}phosphonate; Diethyl {2-[({7-[(4-fluorophenyl)methyl]-4-hydroxy-1-methyl-2-oxo-1,2-dihydro-1,5-naphthyridin-3-yl}carbonyl)amino]ethyl}phosphonate;
Ethyl hydrogen {2-[({7-[(4-fluorophenyl)methyl]-4-hydroxy-1-methyl-2-oxo-1,2-dihydro-1,5-naphthyridin-3-yl}carbonyl)amino]ethyl}phosphonate;
{2-[({7-[(4-Fluorophenyl)methyl]-4-hydroxy-1-methyl-2-oxo-1,2-dihydro-1,5-naphthyridin-3-yl}carbonyl)amino]ethyl}phosphonic acid;
Ethyl P-{2-[({7-[(4-fluorophenyl)methyl]-4-hydroxy-1-methyl-2-oxo-1,2-dihydro-1,5-naphthyridin-3-yl}carbonyl)amino]ethyl}-N-[2-(methyloxy)ethyl]phosphonamidate;
Diethyl {4-[({7-[(4-fluorophenyl)methyl]-4-hydroxy-1-methyl-2-oxo-1,2-dihydro-1,5-naphthyridin-3-yl}carbonyl)amino]butyl}phosphonate;
Ethyl hydrogen {4-[({7-[(4-fluorophenyl)methyl]-4-hydroxy-1-methyl-2-oxo-1,2-dihydro-1,5-naphthyridin-3-yl}carbonyl)amino]butyl}phosphonate;
{4-[({7-[(4-Fluorophenyl)methyl]-4-hydroxy-1-methyl-2-oxo-1,2-dihydro-1,5-naphthyridin-3-yl}carbonyl)amino]butyl}phosphonic acid;
Diethyl {3-[({7-[(4-fluorophenyl)methyl]-4-hydroxy-1-methyl-2-oxo-1,2-dihydro-1,5-naphthyridin-3-yl}carbonyl)amino]propyl}phosphonate;
Ethyl hydrogen {3-[({7-[(4-fluorophenyl)methyl]-4-hydroxy-1-methyl-2-oxo-1,2-dihydro-1,5-naphthyridin-3-yl}carbonyl)amino]propyl}phosphonate;
{3-[({7-[(4-Fluorophenyl)methyl]-4-hydroxy-1-methyl-2-oxo-1,2-dihydro-1,5-naphthyridin-3-yl}carbonyl)amino]propyl}phosphonic acid;
Diethyl {[({7-[(4-fluorophenyl)methyl]-4-hydroxy-1-methyl-2-oxo-1,2-dihydro-1,5-naphthyridin-3-yl}carbonyl)amino]methyl}phosphonate;
Ethyl hydrogen {[({7-[(4-fluorophenyl)methyl]-4-hydroxy-1-methyl-2-oxo-1,2-dihydro-1,5-naphthyridin-3-yl}carbonyl)amino]methyl}phosphonate;
{[({7-[(4-Fluorophenyl)methyl]-4-hydroxy-1-methyl-2-oxo-1,2-dihydro-1,5-naphthyridin-3-yl}carbonyl)amino]methyl}phosphonic acid;
Diethyl {2-[({1-[2-(dimethylamino)-2-oxoethyl]-7-[(4-fluorophenyl)methyl]-4-hydroxy-2-oxo-1,2-dihydro-1,5-naphthyridin-3-yl}carbonyl)amino]ethyl}phosphonate;
Diethyl {[({1 [2-(dimethylamino)-2-oxoethyl]-7-[(4-fluorophenyl)methyl]-4-hydroxy-2-oxo-1,2-dihydro-1,5-naphthyridin-3-yl}carbonyl)amino]methyl}phosphonate;
Diethyl {2-[({1-(4-fluorophenyl)-7-[(4-fluorophenyl)methyl]-4-hydroxy-2-oxo-1,2-dihydro-1,5-naphthyridin-3-yl}carbonyl)amino]ethyl}phosphonate;
Diethyl {[({7-[(4-fluorophenyl)methyl]-4-hydroxy-2-oxo-1-[2-(2-oxo-1-pyrrolidinyl)ethyl]-1,2-dihydro-1,5-naphthyridin-3-yl}carbonyl)amino]methyl}phosphonate;
Ethyl hydrogen {[({7-[(4-fluorophenyl)methyl]-4-hydroxy-2-oxo-1-[2-(2-oxo-1-pyrrolidinyl)ethyl]-1,2-dihydro-1,5-naphthyridin-3-yl}carbonyl)amino]methyl}phosphonate;
{[({7-[(4-Fluorophenyl)methyl]-4-hydroxy-2-oxo-1-[2-(2-oxo-1-pyrrolidinyl)ethyl]-1,2-dihydro-1,5-naphthyridin-3-yl}carbonyl)amino]methyl}phosphonic acid;
4-({2-[7-[(4-Fluorophenyl)methyl]-4-hydroxy-2-oxo-3-{[(phosphonomethyl)amino]carbonyl}-1,5-naphthyridin-1(2H)-yl]ethyl}amino)butanoic acid;
   and pharmaceutically acceptable salts thereof.

Pharmaceutically acceptable salts of the compounds according to the invention include those derived from pharmaceutically acceptable inorganic and organic acids and bases. Examples of suitable acids include hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, ethanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g. sodium), alkaline earth metal (e.g., magnesium), ammonium, NW₄⁺ (wherein W is C₁₋₄ alkyl) and other amine salts. Physiologically acceptable salts of a hydrogen atom or an amino group include salts or organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids. Physiologically acceptable salts of a compound with a hydroxy group include the anion of said compound in combination with a suitable cation such as Na⁺, NH₄⁺, and NW₄⁺ (wherein W is a C₁₋₄alkyl group). Preferred salts include sodium, calcium, potassium, magnesium, choline, meglumine, hydrochloride, and quaternary ammonium.

Other compounds of this invention may be prepared by one skilled in the art following the teachings of the specification coupled with knowledge in the art using reagents that are readily synthesized or commercially available.

Any reference to any of the above compounds also includes a reference to a pharmaceutically acceptable salt thereof.

Salts of the compounds of the present invention may be made by methods known to a person skilled in the art. For example, treatment of a compound of the present invention with an appropriate base or acid in an appropriate solvent will yield the corresponding salt.

Esters of the compounds of the present invention are independently selected from the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted by, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

In such esters, unless otherwise specified, any alkyl moiety present advantageously contains from 1 to 18 carbon atoms, particularly from 1 to 6 carbon atoms, more particularly from 1 to 4 carbon atoms, Any cycloalkyl moiety present in such esters advantageously contains from 3 to 6 carbon atoms. Any aryl moiety present in such esters advantageously comprises a phenyl group.

Ethers of the compounds of the present invention include, but are not limited to methyl, ethyl, butyl and the like.

The compounds of the invention may be further metabolized in vivo to from mono- and di-phosphonic acids which may antiviral activity. These metabolites are also a feature of the present invention.

According to one embodiment of the invention, compounds of formula (I) or salts thereof may be formulated into compositions. In a preferred embodiment, the composition is a pharmaceutical composition, which comprises a compound of formula (I) and pharmaceutically acceptable carrier, adjuvant or vehicle. In one embodiment, the composition comprises an amount of a compound of the present invention effective to treat or prevent viral infection, for example an HIV infection, in a biological sample or in a patient. In another embodiment, compounds of this invention and pharmaceutical compositions thereof, which comprise an amount of a compound of the present innovation effective to inhibit viral replication or to treat or prevent a viral infection or disease or disorder, for example an HIV infection, and a pharmaceutically acceptable carrier, adjuvant or vehicle, may be formulated for administration to a patient, for example, for oral administration.

The present invention features compounds according to the invention for use in medical therapy, for example for the treatment or prophylaxis of a viral infection, for example an HIV infection and associated conditions. The compounds according to the invention are especially useful for the treatment of AIDS and related clinical conditions such as AIDS related complex (ARC), progressive generalized lymphadenopathy (PGL), Kaposi's sarcoma, thromobocytopenic purpura, AIDS-related neurological conditions such as AIDS dementia complex, multiple sclerosis or tropical paraperesis, anti-HIV antibody-positive and HIV-positive conditions, including such conditions in asymptomatic patients.

According to another aspect, the present invention provides a method for the treatment or prevention of the symptoms or effects of a viral infection in an infected patient, for example, a mammal including a human, which comprises administering to said patient a pharmaceutically effective amount of a compound according to the invention. According to one aspect of the invention, the viral infection is a retroviral infection, in particular an HIV infection.

The present invention further includes the use of a compound according to the invention in the manufacture of a medicament for administration to a subject for the treatment of a viral infection, in particular and HIV infection.

The compounds according to the invention may also be used in adjuvant therapy in the treatment of HIV infections or HIV-associated symptoms or effects, for example Kaposi's sarcoma.

The present invention further provides a method for the treatment of a clinical condition in a patient, for example, a mammal including a human which clinical condition includes those which have been discussed hereinbefore, which comprises treating said patient with a pharmaceutically effective amount of a compound according to the invention. The present invention also includes a method for the treatment or prophylaxis of any of the aforementioned diseases or conditions.

Reference herein to treatment extends to prophylaxis as well as the treatment of established conditions, disorders and infections, symptoms thereof, and associated. The above compounds according to the invention and their pharmaceutically acceptable derivatives may be employed in combination with other therapeutic agents for the treatment of the above infections or conditions. Combination therapies according to the present invention comprise the administration of a compound of the present invention or a pharmaceutically acceptable derivative thereof and another pharmaceutically active agent. The active ingredient(s) and pharmaceutically active agents may be administered simultaneously (i.e., concurrently) in either the same or different pharmaceutical compositions or sequentially in any order. The amounts of the active ingredient(s) and pharmaceutically active agent(s) and the relative timings of administration will be selected in order to achieve the desired combined therapeutic effect.

Examples of such therapeutic agents include, but are not limited to, agents that are effective for the treatment of viral infections or associated conditions. Among these agents are (1-alpha, 2-beta, 3-alpha)-9-[2,3-bis(hydroxymethyl)cyclobutyl]guanine [(−)BHCG, SQ-34514, lobucavir]; 9-[(2R,3R,4S)-3,4-bis(hydroxy methyl)-2-oxetanosyl]adenine (oxetanocin-G); acyclic nucleosides, for example acyclovir, valaciclovir, famciclovir, ganciclovir, and penciclovir; acyclic nucleoside phosphonates, for example (S)-1-(3-hydroxy-2-phosphonyl-methoxypropyl) cytosine (HPMPC), [[[2-(6-amino-9H-purin-9-yl)ethoxy]methyl]phosphinylidene]bis(oxymethylene)-2,2-dimethyl propanoic acid (bis-POM PMEA, adefovir dipivoxil), [[(1R)-2-(6-amino-9H-purin-9-yl)-1-methylethoxy]methyl]phosphonic acid (tenofovir), and (R)-[[2-(6-Amino-9H-purin-9-yl)-1-methylethoxy]methyl]phosphonic acid bis-(isopropoxycarbonyloxymethyl)ester (bis-POC-PMPA); ribonucleotide reductase inhibitors, for example 2-acetylpyridine 5-[(2-chloroanilino)thiocarbonyl) thiocarbonohydrazone and hydroxyurea; nucleoside reverse transcriptase inhibitors, for example 3′-azido-3′-deoxythymidine (AZT, zidovudine), 2′,3′-dideoxycytidine (ddC, zalcitabine), 2′,3′-dideoxyadenosine, 2′,3′-dideoxyinosine (ddI, didanosine), 2′,3′-didehydrothymidine (d4T, stavudine), (−)-beta-D-2,6-diaminopurine dioxolane (DAPD), 3′-azido-2′,3′-dideoxythymidine-5′-H-phosphosphonate (phosphonovir), 2′-deoxy-5-iodo-uridine (idoxuridine), (−)-cis-1-(2-hydroxymethyl)-1,3-oxathiolane 5-yl)-cytosine (lamivudine), cis-1-(2-(hydroxymethyl)-1,3-oxathiolan-5-yl)-5-fluorocytosine (FTC), 3′-deoxy-3′-fluorothymidine, 5-chloro-2′,3′-dideoxy-3′-fluorouridine, (−)-cis-4-[2-amino-6-(cyclo-propylamino)-9H-purin-9-yl]-2-cyclopentene-1-methanol (abacavir), 9-[4-hydroxy-2-(hydroxymethyl)but-1-yl]-guanine (H2G), ABT-606 (2HM-H2G) and ribavirin; protease inhibitors, for example indinavir, ritonavir, nelfinavir, amprenavir, saquinavir, fosamprenavir, (R)—N-tert-butyl-3-[(2S,3S)-2-hydroxy-3-N-[(R)-2-N-(isoquinolin-5-yloxyacetyl)amino-3-methylthio-propanoyl]amino-4-phenylbutanoyl]-5,5-dimethyl-1,3-thiazolidine-4-carboxamide (KNI-272), 4R-(4alpha, 5alpha,6beta)]-1,3-bis[(3-aminophenyl)methyl]hexahydro-5,6-dihydroxy-4,7-bis(phenylmethyl)-2H-1,3-diazepin-2-one dimethanesulfonate (mozenavir), 3-[1-[3-[2-(5-trifluoromethylpyridinyl)-sulfonylamino]phenyl]propyl]-4-hydroxy-6alpha-phenethyl-6beta-propyl-5,6-dihydro-2-pyranone (tipranavir), N′-[2(S)-Hydroxy-3(S)-[N-(methoxycarbonyl)-1-tert-leucylamino]-4-phenylbutyl-N^{alpha}-(methoxycarbonyl)-N′-[4-(2-pyridyl)benzyl]-L-tert-leucylhydrazide (BMS-232632), 3-(2(S)-Hydroxy-3(S)-(3-hydroxy-2-methylbenzamido)-4-phenylbutanoyl)-5,5-dimethyl-N-(2-methylbenzyl)thiazolidine-4(R)-carboxamide (AG-1776), N-(2(R)-hydroxy-1(S)-indanyl)-2(R)-phenyl-methyl-4(S)-hydroxy-5-(1-(1-(4-benzo[b]furanylmethyl)-2(S)-N′-(tert-butyl carboxamido)piperazinyl)pentanamide (MK-944A); interferons such as α-interferon; renal excretion inhibitors such as probenecid; nucleoside transport inhibitors such as dipyridamole, pentoxifylline, N-acetylcysteine (NAC), Procysteine, α-trichosanthin, phosphonoformic acid; as well as immunomodulators such as interleukin II or thymosin, granulocyte macrophage colony stimulating factors, erythropoetin, soluble CD₄ and genetically engineered derivatives thereof; non-nucleoside reverse transcriptase inhibitors (NNRTIs), for example nevirapine (BI-RG-587), alpha-((2-acetyl-5-methylphenyl)amino)-2,6-dichloro-benzeneacetamide (loviride), 1-[3-(isopropyl amino)-2-pyridyl]-4-[5-(methanesulfonamido)-1H-indol-2-ylcarbonyl]piperazine monomethanesulfonate (delavirdine), (10R, 11S, 12S)-12-Hydroxy-6,6,10,11-tetramethyl-4-propyl-11,12-dihydro-2H, 6H, 10H-benzo(1,2-b:3, 4-b′:5, 6-b″)tripyran-2-one ((+) calanolide A), (4S)-6-Chloro-4-[1E)-cyclopropyl ethenyl)-3,4-dihydro-4-(trifluoromethyl)-2(1H)-quinazolinone (DPC-083), (S)-6-chloro-4-(cyclopropyl ethynyl)-1,4-dihydro-4-(trifluoromethyl)-2H-3,1-benzoxazin-2-one (efavirenz, DMP 266), 1-(ethoxy methyl)-5-(1-methylethyl)-6-(phenylmethyl)-2,4(1H,3H)-pyrimidinedione (MKC-442), and 5-(3,5-dichloro phenyl)thio-4-isopropyl-1-(4-pyridyl)methyl-1H-imidazol-2-ylmethyl carbamate (capravirine); glycoprotein 120 antagonists, for example PRO-2000, PRO-542 and 1,4-bis[3-[(2,4-dichlorophenyl)carbonyl amino]-2-oxo-5,8-disodiumsulfanyl]naphthalyl-2,5-dimethoxyphenyl-1,4-dihydrazone (FP-21399); cytokine antagonists, for example reticulose (Product-R), 1,1′-azobis-formamide (ADA), 1,11-(1,4-phenylenebis (methylene))bis-1,4,8,11-tetraazacyclotetradecane octahydrochloride (AMD-3100); integrase inhibitors; and fusion inhibitors, for example T-20 and T-1249.

The present invention further includes the use of a compound according to the invention in the manufacture of a medicament for simultaneous or sequential administration with at least another therapeutic agent, such as those defined hereinbefore.

Compounds of the present invention may be administered with an agent known to inhibit or reduce the metabolism of compounds, for example ritonavir. Accordingly, the present invention features a method for the treatment or prophylaxis of a disease as hereinbefore described by administration of a compound of the present invention in combination with a metabolic inhibitor. Such combination may be administered simultaneously or sequentially.

In general a suitable dose for each of the above-mentioned conditions will be in the range of 0.01 to 250 mg per kilogram body weight of the recipient (e.g. a human) per day, preferably in the range of 0.1 to 100 mg per kilogram body weight per day and most preferably in the range 0.5 to 30 mg per kilogram body weight per day and particularly in the range 1.0 to 20 mg per kilogram body weight per day. Unless otherwise indicated, all weights of active ingredient are calculated as the parent compound of formula (I) or (Ia); for salts or esters thereof, the weights would be increased proportionally. The desired dose may be presented as one, two, three, four, five, six or more sub-doses administered at appropriate intervals throughout the day. In some cases the desired dose may be given on alternative days. These sub-doses may be administered in unit dosage forms, for example, containing 10 to 1000 mg or 50 to 500 mg, preferably 20 to 500 mg, and most preferably 50 to 400 mg of active ingredient per unit dosage form.

While it is possible for the active ingredient to be administered alone, it is preferable to present it as a pharmaceutical composition. The compositions of the present invention comprise at least one active ingredient, as defined above, together with one or more acceptable carriers thereof and optionally other therapeutic agents. Each carrier must be acceptable in the sense of being compatible with the other ingredients of the composition and not injurious to the patient.

Pharmaceutical compositions include those suitable for oral, rectal, nasal, topical (including transdermal, buccal and sublingual), vaginal or parenteral (including subcutaneous, intramuscular, intravenous, intradermal, and intravitreal) administration. The compositions may conveniently be presented in unit dosage form and may be prepared by any methods well known in the art of pharmacy. Such methods represent a further feature of the present invention and include the step of bringing into association the active ingredients with the carrier, which constitutes one or more accessory ingredients. In general, the compositions are prepared by uniformly and intimately bringing into association the active ingredients with liquid carriers or finely divided solid carriers or both, and then if necessary shaping the product.

The present invention further includes a pharmaceutical composition as hereinbefore defined wherein a compound of the present invention or a pharmaceutically acceptable derivative thereof and another therapeutic agent are presented separately from one another as a kit of parts.

Compositions suitable for transdermal administration may be presented as discrete patches adapted to remain in intimate contact with the epidermis of the recipient for a prolonged period of time. Such patches suitably contain the active compound 1) in an optionally buffered, aqueous solution or 2) dissolved and/or dispersed in an adhesive or 3) dispersed in a polymer. A suitable concentration of the active compound is about 1% to 25%, preferably about 3% to 15%. As one particular possibility, the active compound may be delivered from the patch by electrotransport or iontophoresis as generally described in Pharmaceutical Research 3(6), 318 (1986).

Pharmaceutical compositions of the present invention suitable for oral administration may be presented as discrete units such as capsules, caplets, cachets or tablets each containing a predetermined amount of the active ingredients; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredients in a free-flowing form such as a powder or granules, optionally mixed with a binder (e.g. povidone, gelatin, hydroxypropylmethyl cellulose), lubricant, inert diluent, preservative, disintegrant (e.g. sodium starch glycollate, cross-linked povidone, cross-linked sodium carboxymethyl cellulose) surface-active or dispersing agent. Molded tablets may be made by molding a mixture of the powdered compound moistened with an inert liquid diluent in a suitable machine. The tablets may optionally be coated or scored and may be formulated so as to provide slow or controlled release of the active ingredients therein using, for example, hydroxypropylmethyl cellulose in varying proportions to provide the desired release profile. Tablets may optionally be provided with an enteric coating, to provide release in parts of the gut other than the stomach.

Pharmaceutical compositions suitable for topical administration in the mouth include lozenges comprising the active ingredients in a flavored base, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions suitable for vaginal administration may be presented as pessaries, tampons, creams, gels, pastes, foams or spray. Pharmaceutical compositions may contain in addition to the active ingredient such carriers as are known in the art to be appropriate.

Pharmaceutical compositions for rectal administration may be presented as a suppository with a suitable carrier comprising, for example, cocoa butter or a salicylate or other materials commonly used in the art. The suppositories may be conveniently formed by admixture of the active combination with the softened or melted carrier(s) followed by chilling and shaping in molds.

Pharmaceutical compositions suitable for parenteral administration include aqueous and nonaqueous isotonic sterile injection solutions which may contain anti-oxidants, buffers, bacteriostats and solutes which render the pharmaceutical composition isotonic with the blood of the intended recipient; and aqueous and non-aqueous sterile suspensions which may include suspending agents and thickening agents; and liposomes or other microparticulate systems which are designed to target the compound to blood components or one or more organs. The pharmaceutical compositions may be presented in unit-dose or multi-dose sealed containers, for example, ampoules and vials, and may be stored in a freeze-dried (lyophilized) condition requiring only the addition of the sterile liquid carrier, for example water for injection, immediately prior to use. Extemporaneous injection solutions and suspensions may be prepared from sterile powders, granules and tablets of the kind previously described.

Unit dosage pharmaceutical compositions include those containing a daily dose or daily subdose of the active ingredients, as hereinbefore recited, or an appropriate fraction thereof.

It should be understood that in addition to the ingredients particularly mentioned above the pharmaceutical compositions of this invention may include other agents conventional in the art having regard to the type of pharmaceutical composition in question, for example, those suitable for oral administration may include such further agents as sweeteners, thickeners and flavoring agents.

The compounds of the present invention may be prepared according to the following reactions schemes and examples, or modifications thereof using readily available starting materials, reagents and conventional synthesis procedures. In these reactions, it is also possible to make use of variants which are known to those of ordinary skill in the art.

The compounds of the present invention are readily prepared by methods outlined in Schemes 1-9 or by methods known to one skilled in the art. Compounds of formula (I) and (Ia) as defined above may be prepared by treating compounds such as 1c with amines (R³NH₂). These and other methods for the conversion of carboxylic esters and acid derivatives to amides are well known to those skilled in the art. For examples, see: March, J., Advanced Organic Chemistry, 4^{th} Edition; John Wiley & Sons, 1992, pp 419-424. Compounds such as 1c are prepared by treating 3-oxopropanoyl derivatives 1b with base (e.g. NaOMe or NaOEt) in protic solvents such as MeOH or EtOH. Oxopropanoyl derivatives 1b may be prepared by reacting amines 1a with malonylchloride derivatives in the presence of base. Alternatively, compounds 1b are prepared by heating a solution of amine 1a with a malonylchloride derivatives in a nonprotic solvent.

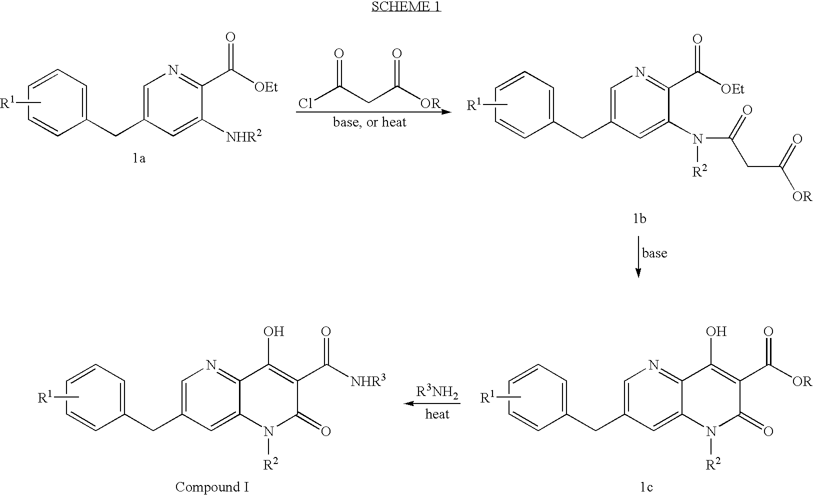

Amines 1a may be prepared by reductive amination of amines 2a with aldehydes and ketones as outlined in Scheme 2. For examples, of reductive amination reactions, see: March, J., Advanced Organic Chemistry, 4^{th} Edition; John Wiley & Sons, 1992, pp 898-900.

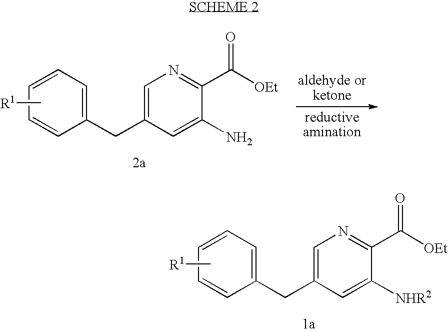

Amines 2a are readily prepared by methods outlined in Scheme 3. Heck reaction of aryl iodides 3a with allyl alcohol generates 3-arylproponals 3b. For examples of Heck reactions in the preparation of 3b, see: March, J., Advanced Organic Chemistry, 4th Edition; John Wiley & Sons, 1992, pp 717-718. Treatment of 3b with formaldehyde in the presence of diethylamine hydrochloride affords requisite 2-benzylpropenals 3c. Reaction of 3c with diethyl 2-aminofumarate provides a pyridine diethyl ester 3d which may be hydrolyzed under basic conditions (e.g. NaOH) to the corresponding pyridine dicarboxylic acid 3e. For synthesis of diethyl 2-aminofumarate, see: Isobe, K.; Mohiri, C.; Sano, H.; Molri, K.; Enomoto, H., Chem. Pharm. Bull., Vol. 37, 1989, pp 3236-3238. Treatment of 3e with acetic anhydride yields the corresponding cyclic anhydride 3f which is treated with EtOH at reflux to generate the pyridine carboxylic acid monoester 3g. Curtius rearrangement of 3g in the presence of t-BuOH yields the BOC-protected 3-aminopyridine derivative 3h which may be deprotected with TFA to afford the desired 3-aminopyridine compound 2a. For an example of a Curtius rearrangement of this type, see: Feiser, M., Reagents for Organic Synthesis, Vol. 11; John Wiley & Sons, 1984, p 222.

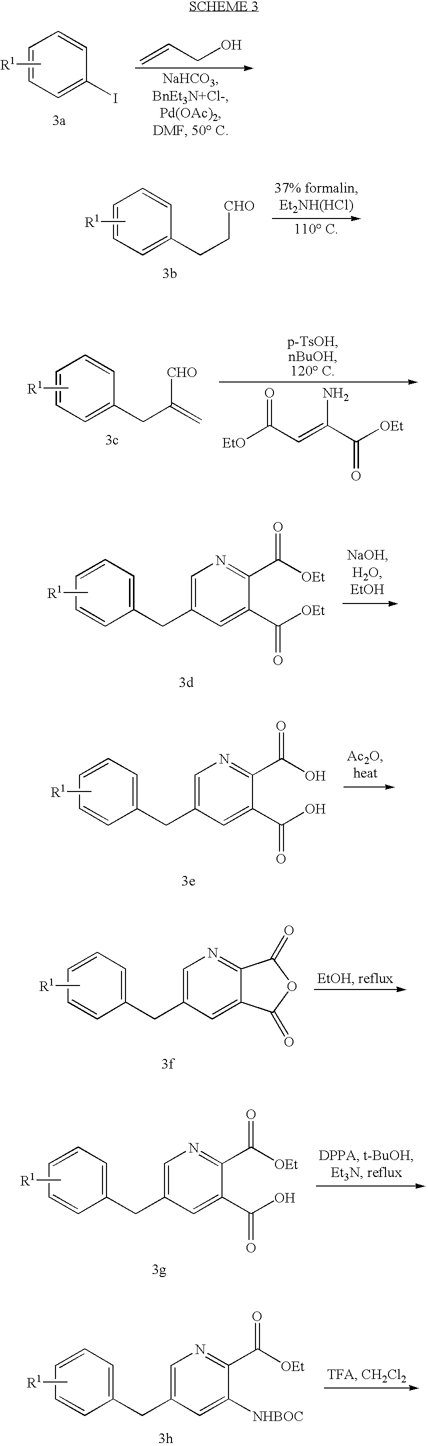

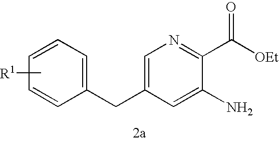

A particularly useful synthesis of a compound similar to 1a (4h) is shown in Scheme 4. Disubstituted pyridines such as 4a can be metallated and reacted with electrophiles such as aldehydes. Conditions for metallation can include by way of example treating a heteroaryl bromide such as 4a with alkyllithium reagents or magnesium in the case of forming Grignard intermediates. The reactive metallated species can then be exposed to an optionally substituted benzaldehyde (4b) at low temperature to form a diaryl carbinol such as 4c. Specific reaction conditions such as temperature and solvent can effect the results of this type of reaction. A particularly useful solvent for this type of chemistry is methyl tert-butyl ether (MTBE). Low temperature condition involve reaction temperature from −78° C. to ambient temperature by way of example. The resultant benzylic alcohol can be converted to the corresponding diarylmethane derivative 4d by way of reduction. Typically conditions for reduction of an alcohol such as 4c involve catalytic hydrogenation or hydride reduction conditions. Catalytic hydrogenation conditions can typically involve the use of Pd/C in an alcoholic solvent or ethyl acetate as an example. A particularly useful reduction protocol well know to those skilled in the art for the reduction of benzylic alcohols involves treatment of 4c with triethylsilane in trifluoroacetic acid. Similarly, triethylsilane and a Lewis acid such as boron trifluoride etherate and the like can also be used in an inert solvent optionally with heating. The methyl ether in 4c is also able to be removed to produce the 2 hydroxypyridine moiety in the same pot as the reduction transformation. In cases where the methyl ether is not sufficiently cleaved, acidic conditions can be used to deblock the phenol. Typically these conditions include a strong acid such as HBr and the like optionally in a solvent such as acetic acid in some cases with heating. Pyridone 4d can be nitrated regioselectively to produce nitrophenol 4e. This type of transformation is commonly known to one skilled in the art, however a particularly useful set of conditions to obtain the desired regiochemistry involve an acidic solvent such as TFA and a nitrating agent such as fuming nitric acid. This material can then be converted to a 2-bromo-pyridine derivative 4f by treatment with phosphorous oxybromide in an inert solvent. Typical solvents of choice include but are not limited to toluene and 1,2-dichloroethane and the like. In some cases the corresponding chloro derivative produced by use of phosphorous oxychloride may also be useful in the same reaction sequence. In some cases a base may be added. Suitable bases may include diethylaniline by means of example. Compounds such as 4f can be converted to a compound such as 4g by carbonylation. Typically these conditions involve the use of a source of palladium (0) and an atmosphere of carbon monoxide optionally at ambient or increased pressures in the presence of a base. In many cases these reactions are best run at elevated temperatures. The catalyst can be tetrakistriphenylphosphine palladium (0) or palladium acetate and the like be way of example. Suitable bases such as triethylamine and the like are typically added. An alcohol is typically added to form the resultant ester. A particularly useful alcohol is methanol. The nitro group in 4g can be reduced to form the aniline 4h using methods well known to those skilled in the art. Typical conditions involve catalytic hydrogenation. Suitable conditions may involve the use of palladium on carbon with an atmosphere of hydrogen at ambient or elevated pressures. In some cases the addition of iron metal can be particularly useful.

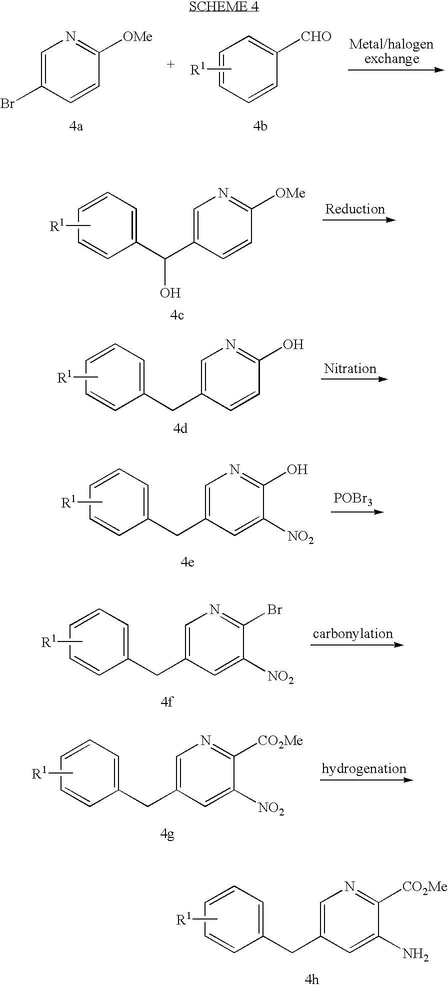

A particularly useful route to produce a compound similar to 1a is shown is Scheme 5. This strategy begins with a 3-fluoro-pyridine such as 5a. It is well precedented in the literature how to oxidize the pyridine 5a to form the corresponding pyridine N-oxide 5b (Sharpless, K. B. et. al. J. Org. Chem. 1998, 63, 1740). The literature method of Sakamoto et. al. (Chem. Pharm. Bull. 1985, 33, 565) can be used to form 2-cyano-3-fluoropyridine 5c by treatment of N-oxide 5b with TMSCN. This method is well known to regioselectively form the 2-nitrile. This material is able to be lithiated according to a modifications of methods described in the literature (WO 2004/019868) and treated with elemental iodine to form the 4-iodo derivative 5d. The 4-iodo derivative 5d can then be rearranged to the 5-iodo derivative 5e again according to a modification of the procedure outlined in the literature (WO 2004/019868). This 5-iodopyridine derivative can be subjected to a palladium mediated cross-coupling known to those skilled in the art as a Negishi-type coupling. Typically these cross-coupling reactions involve the reaction of an aryl halide with a alkyl zinc reagent. In this case reaction of iodide 5e with a benzyl zinc halide in the presence of a catalytic amount of a palladium (0) source resulted in formation of the 5-benzyl derivative 5g. The benzyl zinc halide can be prepared by literature methods or purchased from commercial sources. Typically, the catalyst is Pd(PPh₃)₄ and the like and the solvent is THF. The reaction optionally may be heated. An optionally substituted amine can be used to displace the 3-fluoro substituent in 5g to produce 5 h. Typically this can be done by heating optionally in a microwave a mixture of the amine and 3-fluoropyridine 5g in the amine neat or in an inert solvent to provide the 3-amino-2-cyano derivative 5h. The nitrile functionality may be hydrolyzed under acidic or basic conditions. A particularly useful method involves heating the nitrile in ethanolic sodium hydroxide to give the corresponding carboxylic acid 51. The acid may then be converted to the corresponding ester using several methods well known in the literature. By way of example, particularly useful conditions involve the use of diazomethane, TMS-diazomethane and the like in a solvent such as ether or methanol/benzene respectively. Another particularly useful method for conversion of the acid to ester 5j involves the use of a base and alkylating agent. Typcially, the alkylating agent is methyl iodide and the like and the base is potassium carbonate, triethylamine, sodium hydroxide and the like by way of example. This reaction can be performed optionally in an inert solvent such as DMF and the like.

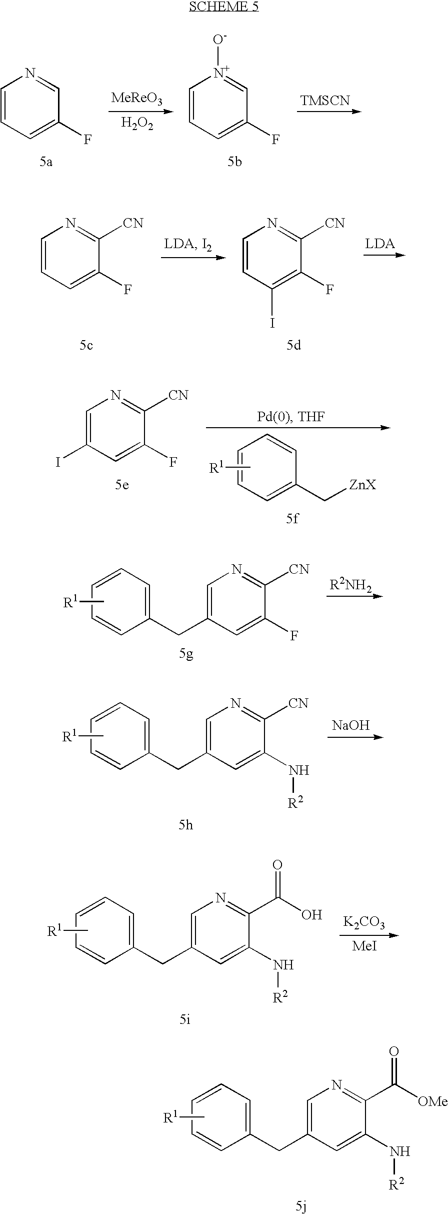

An analogous method to that shown in Scheme 5 can be used to form an intermediate 3,5-dibromo-2-cyanopyridine 6c (Scheme 6). A unique discovery with this system is the selective Negishi coupling to form intermediate 6e with a high level of selectivity. Dibromo derivative 6c can be treated with an optionally substituted benzyl zinc derivative 6d resulting in selective formation of the 5-benzyl product 6e. Typical conditions involved the use of Pd(PPh₃)₄ in an inert solvent such as THF and the like. The 3-bromosubstituent is particularly useful since it is well known that aryl bromides can be used for palladium mediated amination reactions known to those skilled in the art as Buchwald-Hartwig type couplings. This was particularly useful for the formation of compounds where R² was an optionally substituted aryl group however can be used in a general sense to form a wide variety of R² substituted compounds of the formula I. The remainder of the synthesis can proceed as shown in the previous Schemes.

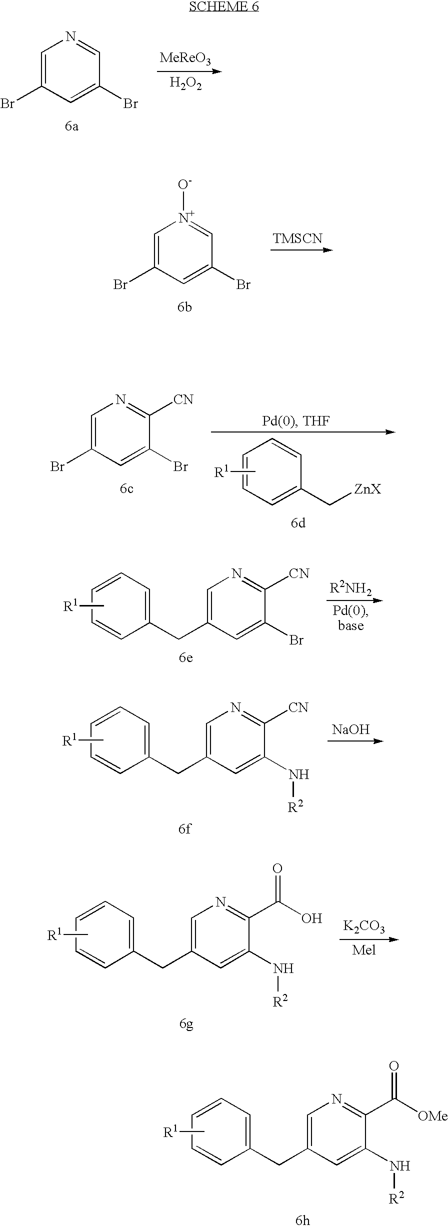

Another noteworthy method to convert a compound such as 2a to a selected group of compounds such as 7a where R² is aryl or heteroaryl involves the use of palladium mediated Buchwald-Hartwig reaction. Typically conditions for this type of reaction involve the use of a source of palladium (0) catalyst, a ligand and a base. By way of example conditions may use palladium acetate and the like as a catalyst. Suitable ligand may include but are not limited to phosphine ligands such as Xantphos. Bases include but are not limited to cesium carbonate and sodium tert-butoxide and the like.

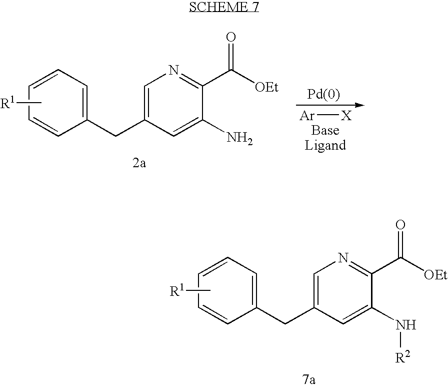

A useful method for conversion of a compound of formula 8a to one of the formula 1c involved the use of an alkylation (Scheme 8). Typically these type of reactions employ a base and an alkylating agent in an inert solvent. By way of example suitable bases include but are not limited to LDA, lithium hexamethyldisilazide, sodium hydride and the like. Alkylating agents include but are not limited to alkyl halides, triflates, mesylates, tosylates and the like.

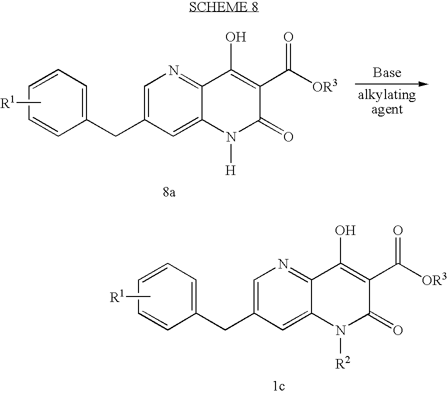

A useful method for conversion of a compound such as 2a to a higher substitued version such as 1a involves the method shown in Scheme 9. The 3 amino group can be activated for alklation by conversion to a trifluoroacetamide or similar group such as shown in structure 9a. Typically this can be formed using trifluoroacetic anhydride or a similar reagent optionally with heating neat or in an inert solvent. Trifluoroacetamide 9a can be alkylated using conditions known to those skilled in the art. Typical conditions may include the use of a base such as potassium carbonate and the like in an inert solvent such as acetonitrile or DMF. Alkylating agents include but are not limited to alkyl halides, triflates, mesylates and the like. Typically removal of the trifluoroacetamide can be accomplished by subjecting 9a to hydrolysis conditions. Suitable conditions typically include heating in an alcohol optionally in the presence of a base.

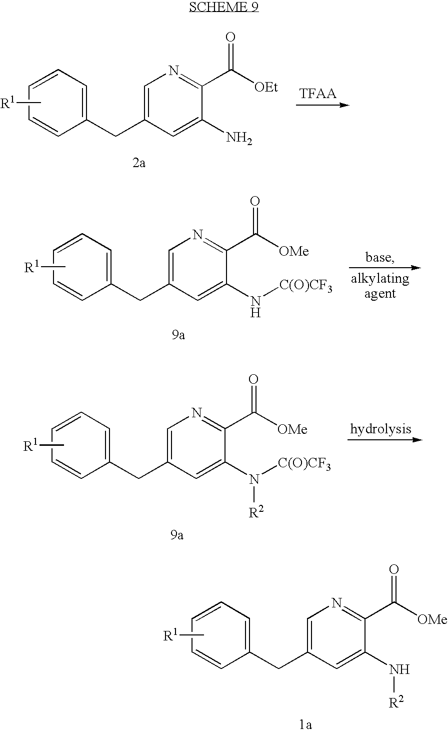

Methods for converting compounds of the formula I to other compounds of formula I are of particular interest. By way of example shown in Scheme 10, a compound of the formula Ia can be converted to a compound of formula Ib by a basic hydrolysis. Typical conditions for such a transformation are well known to one skilled in the art. A compound of formula Ia can be treated with a base such as sodium hydroxide in an aqueous solvent such as water or a mixture of water and an alcoholic solvent such as ethanol. Optionally this reaction can be heated to provide a method for conversion of a compound of formula I to a different compound of formula I.

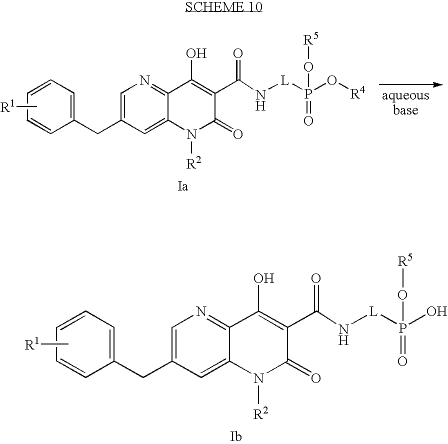

Another useful method for conversion of a compound of formula I to a different compound of formula I is shown in Scheme 11. A compound of formula Ia can be treated with acid to form a compound of formula Ic. Optionally this reaction may require heating and optionally with microwave irradiation at temperatures up to 150° C.

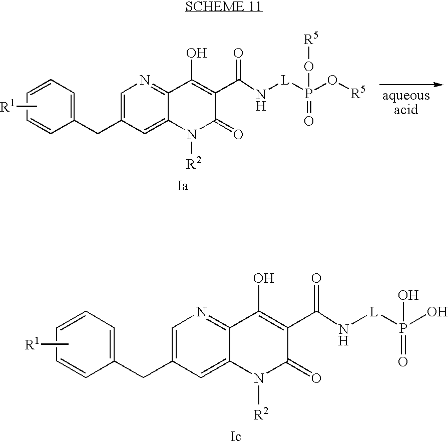

Another useful method for converting a compound of formula I to a different compound of formula I involves treating a compound such as 1b with an amine and a coupling reagent in an inert solvent optionally with a base (Scheme 12) to give a compound of formula Id. Suitable coupling reagents include but are not limited to 0-(7-azabenzotriazol-7-yl-N,N,N′N′-tetramethyluronium hexafluorophosphate (HATU), 1-(3-dimethylaminopropyl)-3-ethylcarbodiimide hydrochloride (EDCI) and the like. Preferred solvents include but are not limited to dichloromethane, tetrahydrofuran and dimethylformamide and the like. Bases can include but are not limited to diisopropyl ethylamine and triethylamine and the like.

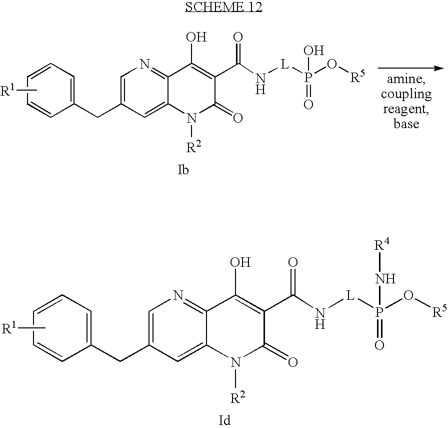

It will be understood by one skilled in the art that the above synthetic steps could be used in different orders to produce compounds of formula I. In particular, the phosphonate group may be manipulated to produce alternative phosphonate derivatives using the methods described above or additional methods known to one skilled in the art for the formation and alteration of phosphoryl compounds. It will be apparent to one skilled in the art that using such methods may be done prior to attachment of the phosphonate optionally containing the linker L group to the rest of the compounds of formula I and corresponding intermediates.

The following examples are intended for illustration only and are not intended to limit the scope of the invention in any way.

### Compound 1: Ethyl 7-(4-fluorobenzyl)-4-hydroxy-2-oxo-1,2-dihydro-1,5-naphthyridine-3-carboxylate

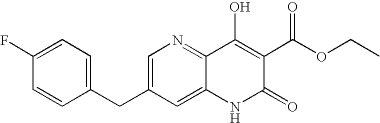

### Step 1: Synthesis of 3-(4-fluorophenyl)propanal

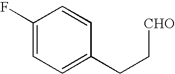

To a mixture of 1-fluoro-4-iodobenzene (300 g, 1.35 mol), benzyltriethylammonium chloride (300 g, 1.35 mol), NaHCO₃ (283 g, 3.4 mol) and allyl alcohol (138 mL, 2.0 mol) in DMF (300 mL) was added palladium acetate (3.0 g, 13.5 mmol). The mixture was heated at 50° C. for 5 h with stirring. Water (1 L) and Et₂O (1 L) were added at rt. After filtration through Celite, the filtrate was extracted with Et₂O. The extracts were washed with H₂O and brine, then dried and concentrated to yield the product: ¹H NMR (CDCl₃) δ 9.81 (1H, s), 7.16 (2H, m), 6.97 (2H, m), 2.93 (2H, t, J=7.5 Hz), 2.77 (2H, t, J=7.5 Hz).

### Step 2: Synthesis of 2-(4-fluorobenzyl)propenal

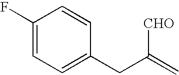

A mixture of 3-(4-fluorophenyl)propanal (205 g, 1.3 mol), diethylamine hydrochloride (148 g, 1.3 mol) and 37% formalin (ca. 1.2 eq.) was heated at 110° C. for 2 h. Water (600 mL) was added and the mixture was extracted with EtOAc. The extract was washed with H₂O and brine, then dried and concentrated to afford the product: ¹H NMR (CDCl₃) δ 9.59 (1H, s), 7.13 (2H, m), 6.98 (2H, m), 6.10 (2H, m), 3.55 (2H, s).

### Steps 3 and 4: Synthesis of 5-(4-fluorobenzyl)-2,3-pyridinedicarboxylic acid

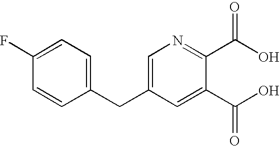

To a solution of diethyl 2-aminofumarate (153 g, 0.81 mol) and p-TsOH.H₂O (1.5 g, 8.1 mmol) in n-BuOH (325 mL) was added 2-(4-fluorobenzyl)propenal (162 g, 0.98 mol) dropwise at 120° C. The mixture was stirred for 17 h at 120° C. and at rt for ca. 24h. The mixture was filtered and concentrated under vacuum to yield the crude diethyl 5-(4-fluorobenzyl)-2,3-pyridinedicarboxylate. This material was dissolved in EtOH (400 mL) and an ice-cold solution of NaOH (88 g, 2.2 mol) in water (300 mL) was added. The mixture was stirred for 2 h at rt. After removal of EtOH in vacuo, water (400 mL) and 6N HCl (200 mL) were added and the mixture was extracted with EtOAc. The EtOAc layer was washed with water and the combined aqueous layers adjusted to pH 2 with 6N HCl (175 mL). The mixture was stirred for 1 h at ice-bath temperature and the product was collected by filtration: ¹H NMR (d₆-DMSO) δ 8.64 (1H, d, J=2 Hz), 8.01 (1H, d, J=2 Hz), 7.32 (2H, dd, J˜9, 6 Hz), 7.12 (2H, t, J˜9 Hz), 4.05 (2H, s); ES⁺MS: 276 (M+H⁺, 100).

### Steps 5 and 6: Synthesis of 5-(4-fluorobenzyl)-2,3-pyridinedicarboxylic acid 2-ethyl ester

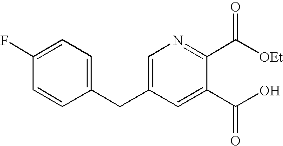

A mixture of 5-(4-fluorobenzyl)-2,3-pyridinedicarboxylic acid (25.3 g, 92 mmol) and Ac₂O (200 mL) was stirred for 3 h at 120° C. The reaction mixture was concentrated in vacuo, dissolved in toluene (200 mL) and re-concentrated in vacuo again to give 5-(4-fluorobenzyl)-2,3-pyridinedicarboxylic anhydride. This material was dissolved in EtOH (200 mL) and the mixture was heated at reflux for 3 h and then stored at rt overnight. The reaction mixture was concentrated in vacuo, dissolved in toluene and concentrated again to afford the product as the major isomer. This material contained ca. 30% of the corresponding 3-ethyl ester 2-carboxylic acid isomer. Major isomer: ¹H NMR (d₆-DMSO) δ 13.5 (1H, br), 8.67 (1H, d, J=2 Hz), 8.06 (1H, d, J=2 Hz), 7.31 (2H, dd, J˜9, 6 Hz), 7.11 (2H, t, J˜9 Hz), 4.25 (2H, q, J=7 Hz), 1.24 (3H, t, J=7 Hz); ES⁺MS: 304 (M+H⁺, 80), 326 (M+Na⁺, 30).

### Step 7: Synthesis of ethyl 5-(4-fluorobenzyl-3-[(tert-butoxy)carbonyl]amino-2-pyridinecarboxylate

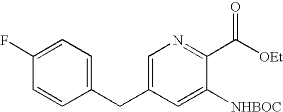

A solution of 5-(4-fluorobenzyl)-2,3-pyridinedicarboxylic acid 2-ethyl ester (28 g, 92 mmol), diphenylphosphoryl azide (29.7 mL, 138 mmol) and Et₃N (38.5 mL, 276 mmol) in t-BuOH (250 mL) was heated at reflux for 5 h and stored at rt for 3 d. After removal of solvent in vacuo, EtOAc was added and the mixture was washed with NH₄Cl solution, NaHCO₃ solution and brine, and then dried and concentrated. The crude material was purified by column chromatography on silica gel eluting with 30% EtOAc/hexanes to afford the product as the major isomer. This material contained ca. 30% of the corresponding 3-ethyl ester 2-(tert-butoxy)carbonylamino isomer. Major isomer: ¹H NMR (d₆-DMSO) δ 9.96 (1H, s), 8.30 (1H, d, J=2 Hz), 8.24 (1H, d, J=2 Hz), 7.27 (2H, m), 7.11 (2H, m), 4.28 (2H, q, J=7 Hz), 4.01 (2H, s), 1.43 (9H, s), 1.28 (3H, t, J=7 Hz); AP⁺MS: 375 (M+H⁺, 100).

### Step 8: Synthesis of ethyl 3-amino-5-(4-fluorobenzyl)-2-pyridinecarboxylate

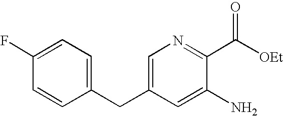

A solution of 5-(4-fluorobenzyl)-3-[(tert-butoxy)carbonyl]amino-2-pyridinecarboxylate (29 g, 77 mmol) in CH₂Cl₂ (200 mL) and trifluoroacetic acid (60 mL) was stirred at rt overnight. The solvent was removed in vacuo and the crude material was dissolved in EtOAc and washed with NaHCO₃ solution and brine. The organic layer was dried, concentrated and chromatographed on silica gel eluting with 20-60% EtOAc/hexanes to yield the product as a light yellow solid: ¹H NMR (d₆-DMSO) δ 7.76 (1H, d, J=1.7 Hz), 7.25 (2H, m), 7.15 (2H, t, J=9 Hz), 6.92 (1H, d, J=1.7 Hz), 6.62 (2H, br s), 4.23 (2H, q, J=7 Hz), 3.87 (2H, s), 1.26 (3H, t, J=7 Hz); AP⁺MS: 275 (M+H⁺, 100); HRMS calcd for C₁₅H₁₅FN₂O₂+H⁺: 275.1196. Found: 275.1206.

### Step 9: Synthesis of ethyl 3-[(3-ethoxy-3-oxopropanoyl)amino]-5-(4-fluorobenzyl)pyridine-2-carboxylate

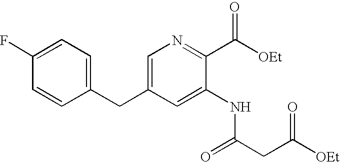

Ethyl 3-chloro-3-oxopropionate (1.32 g, 8.75 mmol) was added to a solution of ethyl 3-amino-5-(4-fluorobenzyl)-2-pyridinecarboxylate (2 g, 7.29 mmol) in DCE (20 mL) and the solution was heated at reflux for 1 h. The solvent was removed in vacuo and silica gel chromatography eluting with 0-5% MeOH/CH₂Cl₂ provide the product as an amber oil: ¹H NMR (CDCl₃) δ 8.96 (1H, br s), 8.32 (1H, d, J=1.8 Hz), 7.17 (2H, dd, J˜9, 6 Hz), 7.00 (2H, t, J˜9 Hz), 4.53 (2H, t, J=7 Hz), 4.29 (2H, t, J=7 Hz), 4.02 (2H, s), 3.54 (2H, s), 1.48 (3H, t, J=7 Hz), 1.33 (3H, t, J=7 Hz).

### Step 10: Synthesis of ethyl 7-(4-fluorobenzyl)-4-hydroxy-2-oxo-1,2-dihydro-1,5-naphthyridine-3-carboxylate

A 2M solution of NaOEt in EtOH (5.87 mL, 11.74 mmol) was added to a solution of ethyl 3-[(3-ethoxy-3-oxopropanoyl)amino]-5-(4-fluorobenzyl)pyridine-2-carboxylate (2.28 g, 5.87 mmol) in EtOH (23 mL) and the mixture was stirred at rt for 1 h. The mixture was neutralized with conc. HCl and concentrated in vacuo. Trituration of the resulting material with a mixture of EtOH and 1:1 brine/water followed by filtration afforded the product as a beige solid: ¹H NMR (d₆-DMSO) δ 11.54 (1H, br s), 8.54 (1H, d, J=1.4 Hz), 7.44 (1H, s), 7.32 (2H, dd, J 8, 6 Hz), 7.17 (2H, t, J˜9 Hz), 4.23 (2H, q, J=7 Hz), 4.12 (2H, s), 1.26 (3H, t, J=7 Hz); HRMS calcd for C₁₈H₁₅FN₂O₄+H⁺: 343.1094. Found: 343.1088.

### EXAMPLE 1

### Diethyl {2-[({7-[(4-fluorophenyl)methyl]-4-hydroxy-2-oxo-[2-dihydro-1,5-naphthyridin-3-yl}carbonyl)amino]ethyl}phosphonate

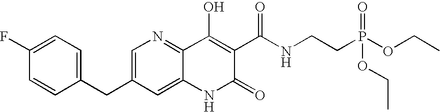

A mixture of ethyl 7-[(4-fluorophenyl)methyl]-4-hydroxy-2-oxo-1,2-dihydro-1,5-naphthyridine-3-carboxylate (70 mg, 0.205 mmol), diethyl (2-aminoethyl)phosphonate oxalate salt (83 mg, 0.307 mmol), and triethylamine (0.06 mL), in ethanol (3 mL) was heated in a microwave reactor at 140° C. for 1200 seconds. Additional triethylamine (0.01 mL) was added and the mixture was heated 1200 seconds more. The suspension was concentrated under reduced pressure, acidified with 1 N HCl and extracted with chloroform. The combined organics were washed with brine, dried over sodium sulfate and concentrated to a yellow solid. The title compound was obtained as a white solid (6 mg, 6%) after purification by HPLC. ¹H NMR (CDCl₃) δ 11.36 (br s, 1H), 10.61 (s, 1H), 8.49 (s, 1H), 7.44 (s, 1H), 7.17-7.13 (m, 2H), 7.01-6.97 (m, 2H), 4.07-4.00 (m, 6H), 3.84 (m, 2H), 2.29 (m, 2H), 1.30 (t, J=7.2 Hz, 6H); HMS calcd' for C₂₂H₂₆FN₃O₆P (M+H): 478.15378, Found: 478.15368.

### Compound 2: Ethyl 7-[(4-fluorophenyl)methyl]-4-hydroxy-1-methyl-2-oxo-1,2-dihydro-1,5-naphthyridine-3-carboxylate

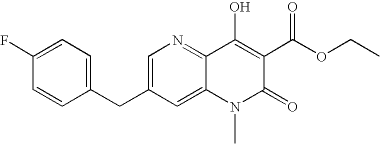

### Step 1: Synthesis of ethyl 5-[(4-fluorophenyl)methyl]-3-(methylamino)-2-pyridinecarboxylate

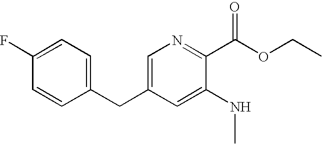

Ethyl 3-amino-5-[(4-fluorophenyl)methyl]-2-pyridinecarboxylate described in compound 1 was reacted with trifluoroacetic anhydride, followed by alkylation with methyl iodide and subsequent deprotection upon heating in the presence of MeOH. ¹H NMR (CDCl₃) δ 7.84 (1H, s), 7.61 (1H, br s), 7.14-7.10 (2H, m), 6.99-6.94 (2H, m), 3.92 (5H, s), 2.83 (3H, d, J=5 Hz); ES⁺MS: 275 (M+H⁺, 100).

### Step 2: Synthesis of ethyl 7-[(4-fluorophenyl)methyl]-4-hydroxy-1-methyl-2-oxo-1,2-dihydro-1,5-naphthyridine-3-carboxylate

A solution of ethyl 5-[(4-fluorophenyl)methyl]-3-(methylamino)-2-pyridinecarboxylate (4.14 g, 0.014 mmol) in DCE (150 mL) was heated to 50° C. and ethyl malonylchloride (3.5 mL, 0.027 mmol) was added streamwise over a period of 3 min.

The reaction mixture was heated at 85° C. for 1 h. After cooling to c.a. 40° C., the reaction was diluted with water and extracted with dichloromethane. The combined organics were washed with a saturated solution of sodium bicarbonate, brine, and dried over sodium sulfate. This material can be purified by silica gel chromatography (0-12% methanol/dichloromethane) or used directly in the next reaction. The resultant material is dissolved in ethanol (100 mL) and placed in an ice bath. Sodium ethoxide (12.34 mL, 29.61 mmol of a 2.4 M solution in ethanol) is added streamwise and the mixture is warmed slowly to ambient temperature and stirred for 1 hour. The suspension is concentrated under reduced pressure and acidified to c.a. pH 3. The resultant solid is then collected by vacuum filtration to give the title compound as a tan solid (4.52 g, 94%, 2 steps). ¹H NMR (CDCl₃) δ 8.51 (1H, s), 7.35 (1H, s), 7.18-7.15 (2H, m), 7.05-7.01 (2H, m), 4.51 (2H, q, J=7 Hz), 4.19 (2H, s), 3.56 (3H, s), 1.23 (3H, t, J=7 Hz); ES+MS: 357 (M+H⁺, 100).

### EXAMPLE 2

### Diethyl {2-[({7-[(4-fluorophenyl)methyl]-4-hydroxy-1-methyl-2-oxo-1,2-dihydro-1,5-naphthyridin-3-yl}carbonyl)amino]ethyl}phosphonate

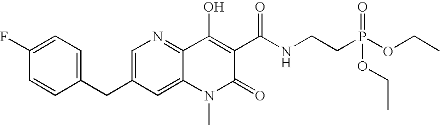

In a similar manner to example 1, the title compound was prepared from ethyl 7-[(4-fluorophenyl)methyl]-4-hydroxy-1-methyl-2-oxo-1,2-dihydro-1,5-naphthyridine-3-carboxylate (33 mg, 0.0926 mmol), diethyl (2-aminoethyl)phosphonate oxalate salt (125 mg, 0.463 mmol) as a white solid (25 mg, 56%) after purification by HPLC. ¹H NMR (CDCl₃) δ 10.38 (br s, 1H), 8.54 (s, 1H), 7.37 (s, 1H), 7.15-7.11 (m, 2H), 7.02-6.97 (m, 2H), 4.14-4.07 (m, 6H), 3.71 (m, 2H), 3.55 (s, 3H), 2.08 (m, 2H), 1.32 (t, J=7.2 Hz, 6H); MS m/z 492 (M+1).

### EXAMPLE 3

### Ethyl hydrogen {2- [({7-[(4-fluorophenyl)methyl]-4-hydroxy-1-methyl-2-oxo-1,2-dihydro-1,5-naphthyridin-3-yl}carbonyl)amino]ethyl}phosphonate

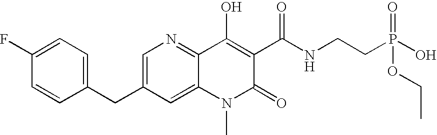

Diethyl {2-[({7-[(4-fluorophenyl)methyl]-4-hydroxy-1-methyl-2-oxo-1,2-dihydro-1,5-naphthyridin-3-yl}carbonyl)amino]ethyl}phosphonate (23 mg, 0.047 mmol) was suspended in 1 N NaOH (3 mL) and the temperature was maintained at 90° C. for 2 hours until a clear solution resulted. The mixture was cooled in an ice bath and acidified with 4 NHCl, extracted several times with chloroform and concentrated under reduced pressure. Purification by HPLC gave the title compound as a white solid (6 mg, 27%). ¹H NMR (CDCl₃) δ 10.38 (br s, 1H), 8.43 (s, 1H), 7.33 (s, 1H), 7.16-7.13 (m, 2H), 7.03-6.98 (m, 2H), 4.13-4.08 (m, 4H), 3.74 (m, 2H), 3.53 (s, 3H), 2.14 (m, 2H), 1.32 (t, J=6.8 Hz, 3H); MS m/z 464 (M+1).

### EXAMPLE 4

### {2-[({7-[(4-Fluorophenyl)methyl]-4-hydroxy-1-methyl-2-oxo-1,2-dihydro-1,5-naphthyridin-3-yl}carbonyl)amino]ethyl}phosphonic acid

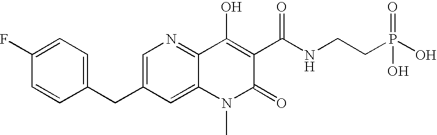

Diethyl {2-[({7-[(4-fluorophenyl)methyl]-4-hydroxy-1-methyl-2-oxo-1,2-dihydro-1,5-naphthyridin-3-yl}carbonyl)amino]ethyl}phosphonate was suspended in 4 N HCl and heated for 1200 seconds in a microwave reactor at 140° C. After cooling to ambient temperature, the white precipitate was collected by vacuum filtration to yield the title compound (13 mg). ¹H NMR (CDCl₃) δ 10.36 (br s, 1H), 8.51 (s, 1H), 7.98 (s, 1H), 7.38-7.34 (m, 2H), 7.13-7.08 (m, 2H), 4.14 (s, 2H), 3.56-3.51 (m, 5H), 1.86 (m, 2H); MS m/z 436 (M+1).

### EXAMPLE 5

### Ethyl P-{2-[({7-[(4-fluorophenyl)methyl]-4-hydroxy-1-methyl-2-oxo-1,2-dihydro-1,5-naphthyridin-3-yl}carbonyl)amino]ethyl}-N-[2-(methyloxy)ethyl]phosphonamidate

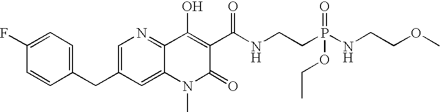

Ethyl hydrogen {2-[({7-[(4-fluorophenyl)methyl]-4-hydroxy-1-methyl-2-oxo-1,2-dihydro-1,5-naphthyridin-3-yl}carbonyl)amino]ethyl}phosphonate (50 mg, 0.108 mmol), [2-(methyloxy)ethyl]amine (0.01 mL, 0.135 mmol), triethylamine (0.04 mL, 0.27 mmol), and HATU (51 mg, 0.135 mmol) were mixed in dimethylformamide for 2 hours. Additional HATU (˜8 eq.) and [2-(methyloxy)ethyl]amine (˜2 eq.) were added until the reaction was judged complete by HPLC. The reaction was quenched with saturated sodium bicarbonate solution, diluted with water and dichloromethane, and extracted with dichloromethane. The combined organics were concentrated under reduced pressure and purified by HPLC to give the title compound as a yellow solid (10 mg, 18% yield). ¹H NMR (CDCl₃) δ 10.40 (br s, 1H), 8.55 (s, 1H), 7.42 (s, 1H), 7.19-7.15 (m, 2H), 7.04-6.99 (m, 2H), 4.14-4.00 (m, 4H), 3.75 (m, 2H), 3.55 (s, 3H), 3.43 (m, 2H), 3.34 (s, 3H), 3.12 (m, 2H), 2.11 (m, 2H), 1.30 (t, J=7.2 Hz, 3H); MS m/z 521 (M+1).

### EXAMPLE 6

### Diethyl {4-[({7-[(4-fluorophenyl)methyl]-4-hydroxy-1-methyl-2-oxo-1,2-dihydro-1,5-naphthyridin-3-}carbonyl amino]butyl}phosphonate

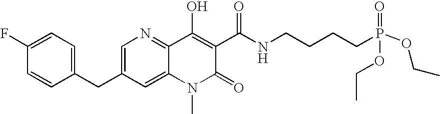

In a manner similar to that described in example 1, the title compound was prepared from ethyl 7-[(4-fluorophenyl)methyl]-4-hydroxy-1-methyl-2-oxo-1,2-dihydro-1,5-naphthyridine-3-carboxylate (50 mg, 0.140 mmol) and diethyl (4-aminobutyl)phosphonate oxalate salt (126 mg) as a white solid (17 mg, 24%) after purification by HPLC. ¹H NMR (CDCl₃) δ 10.19 (m, 1 H0, 8.52 (s, 1H), 7.37 (s, 1H), 7.15-7.11 (m, 2H), 7.01-6.96 (m, 2H), 4.10-4.00 (m, 6H), 3.54 (s, 3H), 3.43 (m, 2H), 1.80-1.66 (m, 6H), 1.28 (t, J=7.2 Hz, 6H); MS mf/z 520 M+1).

### EXAMPLE 7

### Ethyl hydrogen {4-[({7-[(4-fluorophenyl methyl]-4-hydroxy-1-methyl-2-oxo-1,2-dihydro-1,5-naphthyridin-3-yl]carbonyl)amino/butyl}phosphonate

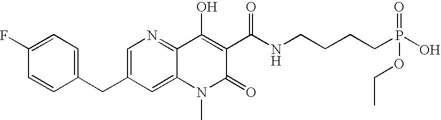

In a manner similar to that described in example 3, the title compound was prepared from diethyl {4-[({7-[(4-fluorophenyl)methyl]-4-hydroxy-1-methyl-2-oxo-1,2-dihydro-1,5-naphthyridin-3-yl}carbonyl)amino]butyl}phosphonate as a white solid (18 mg, 95%). ¹H NMR (CDCl₃) δ 10.17 (br s, 1H), 8.52 (s, 1H), 7.38 (s, 1H), 7.16-7.12 (m, 2H), 7.03-6.98 (m, 2H), 4.11-4.03 (m, 4H), 3.53 (s, 3H), 3.44 (m, 2 H), 1.81-1.74 (m, 6H), 1.31 (t, J=7.2 Hz, 3H); HRMS calcd' for C₂₃H₂₈FN₃O₆P (M+H): 492.1694, Found: 492.1690.

### EXAMPLE 8

### {4-[({7-[(4-Fluorophenyl)methyl]-4-hydroxy-1-methyl-2-oxo-1,2-dihydro-1,5-naphthyridin-3-yl}carbonyl)amino]butyl}phosphonic acid

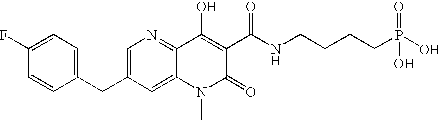

In a manner similar to that described in example 4, the title compound was prepared from diethyl {4-[({7-[(4-fluorophenyl)methyl]-4-hydroxy-1-methyl-2-oxo-1,2-dihydro-1,5-naphthyridin-3-yl}carbonyl)amino]butyl}phosphonate as a white solid. ¹H NMR (CDCl₃) δ 8.66 (br s, 1H), 7.71 (br s, 1H), 7.21-7.14 (m, 2H), 6.99-6.94 (m, 2H), 4.18 (s, 2H), 3.56 (s, 3H), 3.41 (m, 2H), 1.72-1.67 (m, 6H); MS m/z 464 M+1).

### EXAMPLE 9

### Diethyl {3-[({7-[(4-fluorophenyl)methyl]-4-hydroxy-1-methyl-2-oxo-1,2-dihydro-1,5-naphthyridin-3-yl}carbonyl)amino]propyl}phosphonate

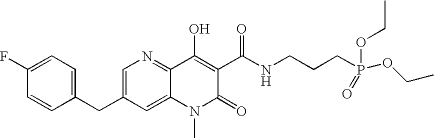

In a manner similar to that described in example 1, the title compound was prepared from ethyl 7-[(4-fluorophenyl)methyl]-4-hydroxy-1-methyl-2-oxo-1,2-dihydro-1,5-naphthyridine-3-carboxylate (50 mg, 0.140 mmol) and diethyl (3-aminopropyl)phosphonate oxalate salt (120 mg) as a white solid (9 mg, 13%) after purification by HPLC. ¹H NMR (CDCl₃) δ 10.24 (br s, 1H), 8.55 (s, 1H), 7.38 (s, 1H), 7.16-7.12 (m, 2H), 7.03-6.98 (m, 2H), 4.12-4.03 (m, 6H), 3.56-3.48 (m, 6H), 1.95-1.76 (m, 4H), 1.30 (t, J=7.2 Hz, 6H); MS m/z 506 M+1).

### EXAMPLE 10

### Ethyl hydrogen {3-[({7-[(4-fluorophenyl)methyl]-4-hydroxy-1-methyl-2-oxo-1,2-dihydro-1,5-naphthyridin-3-yl}carbonyl)amino]propyl}phosphonate

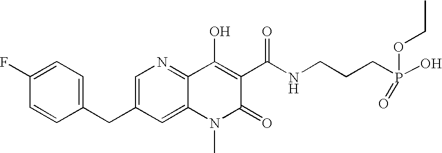

In a manner similar to that described in example 3, the title compound was prepared from diethyl {3-[({7-[(4-fluorophenyl)methyl]-4-hydroxy-1-methyl-2-oxo-1,2-dihydro-1,5-naphthyridin-3-yl}carbonyl)amino]propyl}phosphonate as a white solid. ¹H NMR (CDCl₃) δ 10.23 (br s, 1H), 8.46 (s, 1H), 7.36 (s, 1H), 7.17-7.13 (m, 2H), 7.04-6.99 (m, 2H), 4.13-4.00 (m, 4H), 3.69-3.10 (m, 5H), 2.11-1.81 (m, 4H), 1.31 (m, 3H); MS m/z 478 (M+1).

### EXAMPLE 11

### {3-[({7-[(4-Fluorophenyl)methyl]-4-hydroxy-1-methyl-2-oxo-1,2-dihydro-1,5-naphthyridin-3-yl}carbonyl)amino]propyl}phosphonic acid

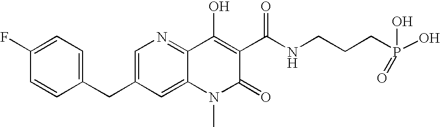

In a manner similar to that described in example 4, the title compound was prepared from diethyl {3-[({7-[(4-fluorophenyl)methyl]-4-hydroxy-1-methyl-2-oxo-1,2-dihydro-1,5-naphthyridin-3-yl}carbonyl)amino]propyl}phosphonate as a white solid. ¹H NMR (methanol-d₄/CDCl3) δ 8.21 (s, 1H), 7.37 (s, 1H), 6.96-6.92 (m, 2H), 6.77-6.73 (m, 2H), 3.90 (s, 2H), 3.35 (s, 3H), 3.25 (m, 2H), 1.67 (m, 2H), 1.50 (m, 2H); HRMS calcd' for C₂₀H₂₂FN₃O₆P (M+H): 450.1230, Found: 450.1244.

### EXAMPLE 12

### Diethyl {[({7-[(4-fluorophenyl)methyl]-4-hydroxy-1-methyl-2-oxo-1,2-dihydro-1,5-naphthyridin-3-yl}carbonyl)amino]methyl}phosphonate

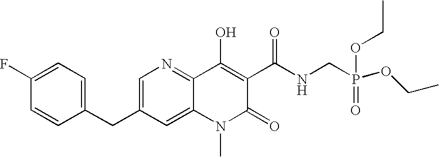

In a manner similar to that described in example 1, the title compound was prepared from ethyl 7-[(4-fluorophenyl)methyl]-4-hydroxy-1-methyl-2-oxo-1,2-dihydro-1,5-naphthyridine-3-carboxylate (50 mg, 0.140 mmol), diethyl (aminomethyl)phosphonate oxalate salt (108 mg, 0.42 mmol), and triethylamine (0.12 mL, 0.84 mmol) as a white solid (17 mg, 26%) after purification by HPLC. ¹H NMR (CDCl₃) δ 10.51 (br s, 1H), 8.60 (s, 1H), 7.43 (s, 1H), 7.18-7.14 (m, 2H), 7.05-7.00 (m, 2H), 4.23-4.14 (m, 6H), 3.94 (m, 2H), 3.59 (s, 3H), 1.35 (t, J=6.8 Hz, 6H); MS m/z 478 (M+1).

### EXAMPLE 13

### Ethyl hydrogen {[({7-[(4-fluorophenyl)methyl]-4-hydroxy-1-methyl-2-oxo-1,2-dihydro-1,5-naphthyridin-3-yl}carbonyl)amino]methyl}phosphonate

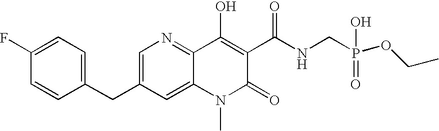

In a manner similar to that described in example 3, the title compound was prepared from diethyl {[({7-[(4-fluorophenyl)methyl]-4-hydroxy-1-methyl-2-oxo-1,2-dihydro-1,5-naphthyridin-3-yl}carbonyl)amino]methyl}phosphonate (8.5 mg, 0.018 mmol) as a white solid (5 mg, 63%) after purification by HPLC. ¹H NMR (CDCl₃) δ 10.49 (br s, 1H), 8.38 (s, 1H), 7.33 (s, 1H), 7.14 (br s, 2H), 7.03-6.98 (m, 2H), 4.17 (br s, 2H), 4.05 (s, 2H), 3.89 (br s, 2H), 3.50 (s, 3H), 1.32 (br s, 3H); MS m/z 450 M+1).

### EXAMPLE 14

### {[({7-[(4-Fluorophenyl)methyl]-4-hydroxy-1-methyl-2-oxo-1,2-dihydro-1,5-naphthyridin-3-yl}carbonyl)amino]methyl}phosphonic acid

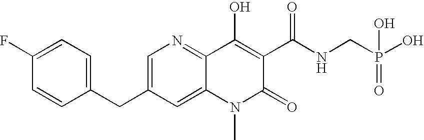

In a manner similar to that described in example 4, the title compound was prepared from diethyl {[({7-[(4-fluorophenyl)methyl]-4-hydroxy-1-methyl-2-oxo-1,2-dihydro-1,5-naphthyridin-3-yl}carbonyl)amino]methyl}phosphonate (10 mg, 0.021 mmol) as a white solid (6.5 mg, 72%). ¹H NMR (methanol-d₄/CDCl3) δ 8.35 (s, 1H), 7.43 (s, 1H), 7.06-7.02 (m, 2H), 6.89-6.84 (m, 2H), 4.01 (s, 2H), 3.67 (d, J=13.6 Hz, 2H), 3.45 (s, 3H); HRMS calcd' for C₁₈H₁₈FN₃O₆P (M+H): 422.0919, Found: 422.0917.

### Compound 3: Ethyl 1-[2-(dimethylamino)-2-oxoethyl]-7-[(4-fluorophenyl)methyl]-4-hydroxy-2-oxo-1,2-dihydro-1,5-naphthyridine-3-carboxylate

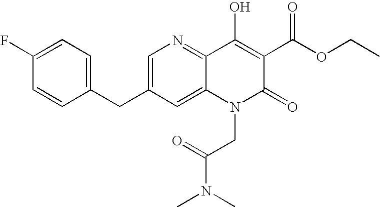

### Step 1: Synthesis of (4-fluorophenyl)[6-(methyloxy)-3-pyridinyl]methanol

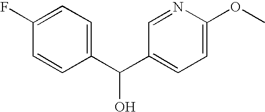

A 2.5M solution of n-BuLi in THF (181 mL, 0.452 mol) was added slowly to a stirred solution of 5-bromo-2-(methyloxy)pyridine (85 g, 0.452 mol) in THF (500 mL) cooled to −65° C. The internal temperature was maintained at or below −55° C. during the addition; when the addition was complete, a solution of 4-fluorobenzaldehyde (51 g, 0.411 mol) in THF (120 mL) was added slowly maintaining the temperature at or below −50° C. Saturated ammonium chloride solution (200 mL) was added and the mixture was warmed to −15° C. and concentrated at reduced pressure. The mixture was diluted with EtOAc (1 L) and washed twice with saturated ammonium chloride solution. The aqueous layers were back-extracted with EtOAc and the combined organic layers were washed with brine, dried and concentrated to afford the product as a light amber oil: ¹H NMR (CDCl₃) δ 8.14 (1H, d, J=2.5 Hz), 7.54 (1H, dd, J=8.6, 2.5 Hz), 7.36 (2H, dd, =8.6, 5.4 Hz), 7.05 (2H, m), 6.73 (1H, d, J=8.6 Hz), 5.82 (1H, s), 3.94 (3H, s), 2.40 (1H, br).

### Steps 2-3: Synthesis of 5-[(4-fluorophenyl)methyl]-2(1H)-pyridinone

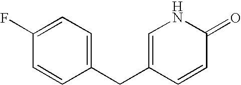

A stirred mixture of (4-fluorophenyl)[6-(methyloxy)-3-pyridinyl]methanol (293 g, 1.26 mol), DCE (650 mL), TFA (650 mL) and triethylsilane (650 mL) was heated at reflux for 5 h; the DCE was removed by distillation and glacial acetic acid (250 mL, 4.4 mol) and 48% HBr (250 mL, 2.2 mol) were added. The resulting solution was heated at reflux for 6.5 h during which time additional 48% HBr (100 mL, 0.88 mol) was added. The mixture was partially concentrated at reduced pressure and the remaining bi-phasic mixture was separated. The upper phase, containing silane bi-products from the previous step, was discarded and the lower phase was cooled in an ice bath and neutralized with 4N NaOH solution to pH 8-9. The resulting precipitate was collected by filtration, washed with water and dried in a vacuum oven to afford the product as a white solid: ¹H NMR (d₆-DMSO) δ 11.39 (1H, br), 7.23 (4H, m), 7.08 (2H, m), 6.24 (1H, d, J=9 Hz), 3.62 (2H, s); ES⁺MS: 204 (+H⁺, 100).

### Step 4: Synthesis of 5-[(4-fluorophenyl methyl]-3-nitro-2(1H)-pyridinone

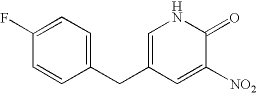

A solution of 90% HNO₃ (57 mL, 1.22 mol) was added slowly to a stirred solution of 5-[(4-fluorophenyl)methyl]-2(1H)-pyridinone (249 g, 1.22 mol) in TFA (750 mL). The solution was heated at 75° C. for 2 h during which time additional 90% HNO₃ (25 mL, 0.5 mol) was added. Water (1 L) was added slowly and most of the TFA was removed by distillation. The mixture was allowed to cool to rt and the product was isolated by filtration as a yellow solid: ¹H NMR (d₆-DMSO) δ 12.76 (1H, br), 8.31 (1H, d, J=2.4 Hz), 7.80 (1H, d, J=2.4 Hz), 7.30 (2H, dd, J=8.6, 5.7 Hz), 7.11 (2H, t, J=8.6 Hz), 3.76 (2H, s); ES⁺MS: 249 (M+H⁺, 100).

### Step 5: Synthesis of 2-bromo-5-[(4-fluorophenyl)methyl]-3-nitropyridine

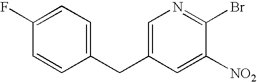

A solution of POBr₃ (227 g, 0.79 mol) in toluene (900 mL) was added slowly to a stirred suspension of 5-[(4-fluorophenyl)methyl]-3-nitro-2(1H)-pyridinone (179 g, 0.72 mol) in toluene (900 mL). The mixture was heated to reflux; then cooled to rt and DMF (56 mL, 0.72 mol) was added slowly; the mixture was again heated to reflux and then allowed to cool to rt overnight. After cooling the mixture in an ice-bath, water (500 mL) was added slowly followed by dropwise addition 4 N NaOH (450 mL, 1.76 mol). All insoluble material was removed by filtration and the two liquid phases were separated. The organic layer was concentrated at reduced pressure to afford the product as a beige solid: ¹H NMR (d₆-DMSO) δ 8.62 (1H, d, J=2 Hz), 8.37 (1H, d, J=2 Hz), 7.33 (2H, m), 7.12 (2H, m), 4.05 (2H, s); ES⁺MS: 313 (M+H⁺, 100), 311 (M+H⁺, 100).

### Step 6: Synthesis of methyl 5-[(4-fluorophenyl)methyl]-3-nitro-2-pyridinecarboxylate

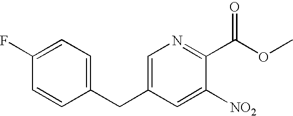

A mixture of bromo-5-[(4-fluorophenyl)methyl]-3-nitropyridine (206 g, 0.66 mol), TEA (230 mL, 1.66 mol), (o-tol)₃P (5 g, 16.4 mmol), and Pd(OAc)₂ (3.7 g, 16.6 mmol) in MeOH (2 L) was heated at 60-65° C. under a CO_{(g)} atmosphere for 33 h. During this time additional (o-tol)₃P (5 g, 16.4 mmol), and Pd(OAc)₂ (5.2 g, 23 mmol) were added. The mixture was filtered through celite, concentrated at reduced pressure, reconstituted in EtOAc and washed with saturated NaHCO₃ solution and brine. The organic phase was dried and concentrated to provide the product as a dark oil: ¹H NMR (d₆-DMSO) δ 8.71 1H, d, J=1.5 Hz), 8.03 (1H, d, J=1.5 Hz), 7.14 (2H, m), 7.06 (2H, m), 4.10 (2H, s), 3.99 (3H, s); ES⁺MS: 291 (+H⁺, 100).

### Step 7: Synthesis of methyl 3-amino-5-[(4-fluorophenyl)methyl]-2-pyridinecarboxylate

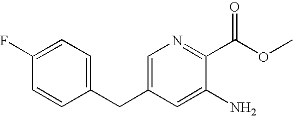

A mixture of methyl 5-[(4-fluorophenyl)methyl]-3-nitro-2-pyridinecarboxylate (200 g, 0.66 mol) and Degussa 10% Pd on carbon (50% by weight water, 20g) in THF (1.5 L) was stirred under an atmosphere of H₂ for 2 d. The mixture was filtered through celite and the filtrate was re-subjected to similar hydrogenation conditions with 10% Pd on carbon (30g) at 45° C. for 7 d. During this time, conc. HCl (14 mL, 0.17 mol) in MeOH (75 mL) and 10% Pd on carbon (18g) were added in approximately three portions each. The mixture was filtered through celite, concentrated at reduced pressure, reconstituted in CH₂Cl₂ and washed with saturated NaHCO₃ solution. The organic phase was concentrated and the resulting material was triturated with EtOAc to provide the product as an off-white solid: ¹H NMR (CDCl₃) δ 7.94 (1H, d, J=1.5 Hz), 7.11 (2H, m), 6.98 (2H, m), 6.72 (1H, 1.5 Hz), 5.67 (2H, br s), 3.95 (3H, s), 3.89 (2H, s); ES⁺MS: 261 (M+H⁺, 100).

### Steps 8-10: Synthesis of methyl 3-{[2-(dimethylamino)-2-oxoethyl]amino}-5-[(4-fluorophenyl)methyl]-2-pyridinecarboxylate

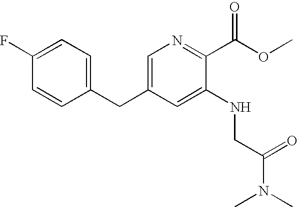

A stirring suspension of methyl 3-amino-5-[(4-fluorophenyl)methyl]-2-pyridinecarboxylate (60 g, 0.23 mol) in i-PrOAc (400 mL) was heated to 30° C. and trifluoroacetic anhydride (35.3 mL, 0.254 mol) was added dropwise. The reaction mixture was stirred 15 min at 30° C.; then cooled to rt and quenched slowly with 0.6 M NaHCO₃ solution (512 mL, 0.32 mol). The resulting biphasic mixture was separated and the organic phase was washed twice with water; then diluted with CH₃CN (700 mL) and distilled to about half its initial volume. To the remaining solution (ca. 600 mL) was added K₂CO₃ (34.6 g, 0.255 mol), NaI (5.18 g, 34.6 mol) and 2-chloro-N,N-dimethylacetamide (26.1 mL, 0.254 mol) and the resulting mixture was heated to 80° C. for 3.5 h. The reaction was cooled to 60° C., diluted with MeOH (200 mL), heated at reflux for 2 h and then distilled to approximately half its original volume. The remaining mixture was cooled to 37° C. and water (650 mL) was added over 2 h with gradual cooling to 15° C. A precipitate formed which was collected by filtration and washed with water. Drying of the filter cake in a vacuum oven afforded the product as an off-white solid: ¹H NMR (CDCl₃) δ 8.56 (1H, br), 7.89 (1H, s), 7.12 (2H, dd, J=8.5, 5.5 Hz), 6.98 (2H, t, J=8.5 Hz), 6.68 (1H, s), 3.96 (3H, s), 3.93 (2H, s), 3.88 (2H, d, J=4.2 Hz), 3.02 (3H, s), 3.01 (3H, s).

### Steps 11-12: Synthesis of ethyl 1-[2-(dimethylamino)-2-oxoethyl]-7-[(4-fluorophenyl)methyl]-4-hydroxy-2-oxo-1,2-dihydro-1,5-naphthyridine-3-carboxylate

Ethyl malonyl chloride (27 mL, 0.21 mol) was added slowly to a solution of methyl 3-{[2-(dimethylamino)-2-oxoethyl]amino}-5-[(4-fluorophenyl)methyl]-2-pyridinecarboxylate (65.8 g, 0.19 mol) in DCE (350 mL) at rt. The mixture was heated at reflux for 3 h during which time additional ethyl malonyl chloride (10 mL, 78 mmol) was added. When the reaction was complete, the mixture was cooled to rt; washed three times with 0.8 M NaHCO₃ solution and once with water. The organic phase was diluted with EtOH (50 mL), distilled to approximately 30% of its original volume and cooled to rt. A solution of 2.68 M NaOEt in EtOH (70 mL, 0.188 mol) was added and after stirring 10 min at rt, the mixture was acidified with 1N HCl (190 mL, 190 mmol) and diluted with EtOH (600 mL). The mixture was heated to 70° C.; then cooled to 50° C. and filtered. The filter cake was washed with water and dried in a vacuum oven to afford the product as a white solid: ¹H NMR (d₆-DMSO) δ 8.44 (1H, d, J=1 Hz), 7.67 (1H, s), 7.31 (2H, dd, J=8.7, 5.6 Hz), 7.12 (2H, t, J=8.7 Hz), 5.04 (2H, s), 4.21 (2H, q, J=7 Hz), 4.11 (2H, s), 3.10 (3H, s), 2.80 (3H, s), 1.23 (3H, t, J=7 Hz); ES⁻MS: 426 (M−1, 100).

### EXAMPLE 15

### Diethyl {2-[({2-(dimethylamino)-2-oxoethyl]-7-[(4-fluorophenyl)methyl]-4-hydroxy-2-oxo-1,2-dihydro-1,5-naphthyridin-3-yl}carbonyl)amino]ethyl}phosphonate

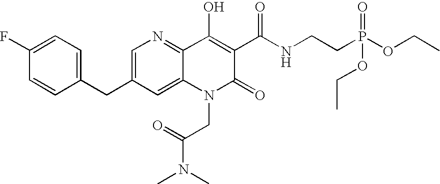

The title compound was prepared in a manner similar to that described in example 1 from ethyl 1-[2-(dimethylamino)-2-oxoethyl]-7-[(4-fluorophenyl)methyl]-4-hydroxy-2-oxo-1,2-dihydro-1,5-naphthyridine-3-carboxylate (35 mg, 0.082 mmol), (2-aminoethyl)phosphonate oxalate salt (67 mg, 0.246 mmol), and triethylamine (0.07 mL) Purification by HPLC gave a white solid (15 mg, 34%). ¹H NMR (CDCl₃) δ 10.21 (br s, 1H), 8.51 (s, 1H), 7.14-7.10 (m, 2H), 7.02-6.98 (m, 3H), 4.93 (s, 2H), 4.14-4.05 (m, 6H), 3.69 (m, 2H), 3.10 (s, 3H), 2.94 (s, 3H), 2.11 (m, 2H), 1.31 (t, J=7.2 Hz, 6H); MS m/z 563 (M+1).

### EXAMPLE 16

### Diethyl {[({1-[2-(dimethylamino)-2-oxoethyl]-7-[(4-fluorophenyl)methyl]-4-hydroxy-2-oxo-1,2-dihydro-1,5-naphthyridin-3-yl}carbonyl)amino]methyl}phosphonate

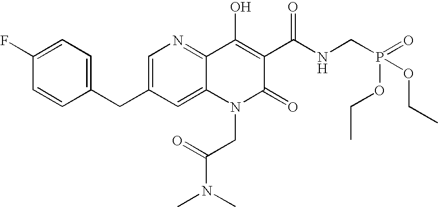

In a manner similar to that described in example 1, the title compound was prepared from ethyl 1-[2-(dimethylamino)-2-oxoethyl]-7-[(4-fluorophenyl)methyl]-4-hydroxy-2-oxo-1,2-dihydro-1,5-naphthyridine-3-carboxylate (50 mg, 0.117 mmol), and diethyl (aminomethyl)phosphonate oxalate salt (90 mg, 0.351 mmol) as a white solid (4 mg, 6%) after purification by HPLC. ¹H NMR (CDCl₃) δ 10.31 (br s, 1H), 8.54 (s, 1H), 7.15-7.11 (m, 2H), 7.05-6.99 (m, 3H), 4.95 (s, 2H), 4.21-4.10 (m, 6H), 3.90 (m, 2H), 3.11 (s, 3H), 2.95 (s, 3H), 1.33 (t, J=6.8 Hz, 6H); MS m/z 549 (M+1).

### Compound 4: Ethyl 7-(4-fluorobenzyl)-1-(4-fluorophenyl)-4-hydroxy-2-oxo-1,2-dihydro-1,5-naphthyridine-3-carboxylate

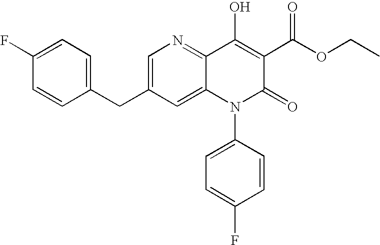

### Step 1: 3,5-dibromo-2-pyridinecarbonitrile

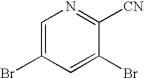

3,5-dibromopyridine (30.5 g, 0.12 mol) was dissolved in dichloromethane (80 mL) and methlytrioxorhenium (150 mg. 0.603 mmol) was added. 30% hydrogen peroxide (27 mL) was added slowly over 5 minutes and the mixture was stirred at ambient temperature for 3 hours. An additional 40 mL of hydrogen peroxide was added and the reaction was stirred 16 hours. Manganese dioxide (100 mg) was added and the suspension was stirred 40 minutes. The mixture was extracted with dichloromethane, dried over sodium sulfate, filtered, and concentrated under reduced pressure. Ethyl acetate was added and the suspension was refluxed for 30 minutes until solids dissolved and then the mixture was allowed to cool to ambient temperature and aged 48 hours. 3,5-dibromopyridine 1-oxide (25.09 g, 82% yield) was collected by vacuum filtration as pale yellow needles. ¹H NMR (CDCl₃) δ 8.27 (s, 2H), 7.56 (s, 1H).

3,5-dibromopyridine 1-oxide (25.09 g, 0.099 mol) was dissolved in acetonitrile (200 mL) and triethylamine (28 mL, 0.198 mol) and trimethylsilylcyanide (40 mL, 0.297 mol) were added. The reaction was stirred 16 hours and then diluted with dichloromethane, aqueous sodium carbonate, water, and then filtered through Celite eluting with dichloromethane. The mixture was extracted with dichloromethane and purified by silica gel chromatography (3:2 hexanes:ethyl acetate gradient elution) to afford 3,5-dibromo-2-pyridinecarbonitrile (18.98 g, 73% yield) as a tan solid. ¹H NMR (CDCl₃) δ 8.68 (d, J=2 Hz, 1H), 8.20 (d, J=2 Hz, 1H); MS m/z 262 (M+1).

### Step 2: 3-bromo-5-[(4-fluorophenyl)methyl]-2-pyridinecarbonitrile

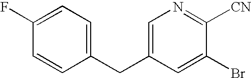

3,5-dibromo-2-pyridinecarbonitrile (4.52 g, 15.34 mmol) was dissolved in tetrahydrofuran (75 mL) and palladium tetrakis(triphenylphosphine) (0.887 g, 0.767 mmol) and 4-fluorobenzyl zinc chloride (46.02 mL, 0.5 M in tetrahydrofuran) were added. The mixture was heated at 85° C. for 1 hour. An additional 12.3 mL of 4-fluorobenzyl zinc chloride was added and the reaction was heated 40 minutes and allowed to cool to ambient temperature. The mixture was diluted with water and ethyl acetate and several drops of 1 N hydrochloric acid were added and the reaction was extracted with ethyl acetate. The organics were washed with brine, dried over sodium sulfate and concentrated under reduced pressure. Purification by silica gel chromatography (0-100% ethyl acetate/hexanes gradient elution) gave 3-bromo-5-[(4-fluorophenyl)methyl]-2-pyridinecarbonitrile (2.54 g, 57% yield) as a pale yellow solid. ¹H NMR (CDCl₃) δ 8.48 (s, 1H), 7.74 (s, 1H), 7.15-7.11 (m, 2H), 7.07-7.02 (m, 2H), 4.02 (s, 2H); MS m/z 292 (M+1).

### Step 3: 3-[(4-fluorophenyl)amino]-5-[(4-fluorophenyl)methyl]-2-pyridinecarbonitrile

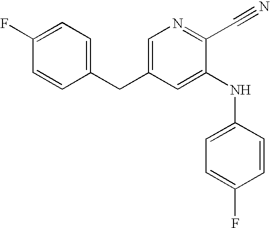

3-Bromo-5-[(4-fluorophenyl)methyl]-2-pyridinecarbonitrile (5.34 g, 18.35 mmol), cesium carbonate (8.37 g, 25.69 mmol), BINAP (860 mg, 1.38 mmol), palladium acetate (206 mg, 0.918 mmol), toluene (200 mL), and 4-fluoroaniline (2.08 mL, 22.02 mmol) were heated at reflux for 3 hours. The reaction was cooled to ambient temperature, filtered through Celtie, eluting with dichloromethane. Silica gel chromatography (0-12% methanol/dichloromethane) gave the title compound as a yellow solid (3.57 g, 61%). ¹H NMR (CDCl₃) δ 7.94 (s, 1H), 7.10-6.94 (m, 8H), 6.24 (s, 1H), 3.85 (s, 2H); MS m/z 322 (M+1).

### Step 4: 3-[(4-fluorophenyl)amino]-5-[(4-fluorophenyl)methyl]-2-pyridinecarboxylic acid

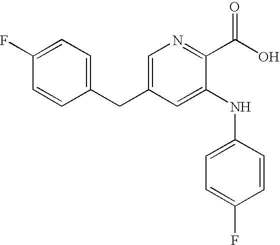

3-[(4-fluorophenyl)amino]-5-[(4-fluorophenyl)methyl]-2-pyridinecarbonitrile (3.57 g, 11.12 mmol), 50 wt. % sodium hydroxide (50 mL), and ethanol (20 mL) were heated at reflux until the reaction was judged complete by HPLC. The reaction was cooled to ambient temperature and filtered through a glass frit. The collected solids were suspended in water and concentrated HC1 was added to make the mixture acidic, then the aqueous layer was extracted several times with ethyl acetate and chloroform. The filtrate from above was acidified the conc. HC1 and extracted with ethyl acetate. The organic layers were combined and azeotroped with methanol to yield the title compound as a yellow solid (3.71 g, 98%). ¹H NMR (DMSO-d₆) δ 9.67 (s, 1H), 7.89 (s, 1H), 7.37 (s, 1H), 7.25-7.06 (m, 8H), 3.93 (s, 2H); MS m/z 341 (M+1).

### Step 5: Methyl 3-[(4-fluorophenyl)amino]-5-[(4-fluorophenyl)methyl]-2-pyridinecarboxylate

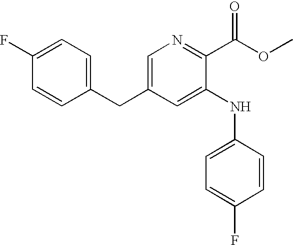

3-[(4-Fluorophenyl)amino]-5-[(4-fluorophenyl)methyl]-2-pyridinecarboxylic acid (3.71 g, 10.91 mmol), DMF (100 mL), and potassium carbonate (3.31 g 24.01 mmol) were stirred at ambient temperature for 1 hour. Water and ethyl acetate were added and the reaction was extracted with ethyl acetate. The combined organics were concentrated under reduced pressure and azeotroped with toluene to yield the title compound (2.08 g, 54%) as a yellow solid. ¹H NMR (DMSO-d₆) δ 9.30 (s, 1H), 7.94 (s, 1H), 7.13-6.92 (m, 8H), 3.98 (s, 3H), 3.84 (s, 2H); MS m/z 355 (M+1).

### Step 6: Ethyl 7-(4-fluorobenzyl)-1-(4-fluorophenyl)-4-hydroxy-2-oxo-1,2-dihydro-1,5-naphthyridine-3-carboxylate

In a manner similar to that described in example 1, the title compound was prepared from methyl 3-[(4-fluorophenyl)amino]-5-[(4-fluorophenyl)methyl]-2-pyridinecarboxylate (2 g, 5.65 mmol) as a white solid (2.38 g, 98%, 2 steps). ¹H NMR (methanol-d₄/CDCl₃) δ 8.19 (s, 1H), 7.17-7.06 (m, 4H), 6.94-6.83 (m, 4H), 6.58 (s, 1H), 4.26 (q, J=7.2 Hz, 2H), 3.81 (s, 2H), 1.27 (t, J=7.2 Hz, 3H); MS m/z 459 (M+23).

### EXAMPLE 17

### Diethyl {2-[({1 (4-fluorophenyl)-7-[(4-fluorophenyl)methyl]-4-hydroxy-2-oxo-1,2-dihydro-1,5-naphthyridin-3-yl}carbonyl)amino]ethyl}phosphonate

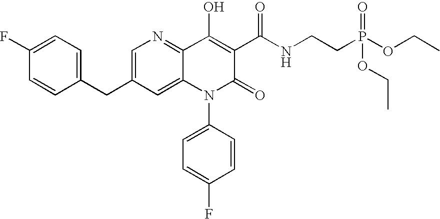

In a manner similar to that described in example 1, the title compound was prepared from ethyl 1-(4-fluorophenyl)-7-[(4-fluorophenyl)methyl]-4-hydroxy-2-oxo-1,2-dihydro-1,5-naphthyridine-3-carboxylate (23 mg, 0.055 mmol) and diethyl (2-aminoethyl)phosphonate oxalate salt (45 mg, 0.165 mmol) as a white solid (15 mg, 48%) after purification by HPLC. ¹H NMR (CDCl₃) δ 10.11 (br s, 1H), 8.51 (s, 1H), 7.28-7.24 (m, 2H), 7.18-7.15 (m, 2H), 7.01-6.90 (m, 4H), 6.70 (s, 1H), 4.11-4.04 (m, 4H), 3.93 (s, 2H), 3.66 (m, 2H), 2.08 (m, 2H), 1.29 (t, J=7.2 Hz, 6H); MS m/z 572M+1).

### Compound 5: Ethyl 7-(4-fluorobenzyl)-4-hydroxy-2-oxo-1-[2-(2-oxopyrrolidin-1-yl)ethyl]-1,2-dihydro-1,5-naphthyridine-3-carboxylate

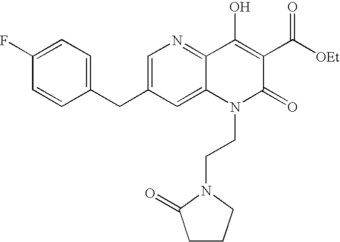

### Step 1: Synthesis of (2-oxopyrrolidin-1-yl)acetaldehyde

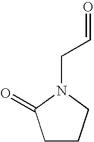

Oxalyl chloride (0.87 mL, 10 mmol) was added dropwise to a solution of DMSO (0.71 mL, 10 mmol) in CH₂Cl₂ (7 mL) cooled to −78° C. After stirring 15 min at this temperature, 1-(2-hydroxyethyl)-2-pyrrolidinone (1g, 7.7 mmol) was added dropwise. The mixture was stirred 30 min at −78° C. and Et₃N (2.8 mL, 20 mmol) was added dropwise. After allowing the reaction to warm to rt, a solution of NaHCO₃ was added and the mixture was extracted with CH₂Cl₂ (6×). The combined organic layers were dried and concentrated to give the product as an oil: ¹H NMR (CDCl₃) δ 9.60 (1H, s), 4.16 (2H, s), 3.46 (2H, t, J=7 Hz), 2.45 (2H, t, J=8 Hz), 2.11 (2H, m).

### Step 2: Synthesis of ethyl 5-(4-fluorobenzyl)-3-{[2-(2-oxopyrrolidin-1-yl)ethyl]amino}pyridine-2-carboxylate

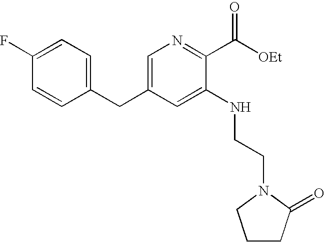

(2-Oxopyrrolidin-1-yl)acetaldehyde and ethyl 3-amino-5-(4-fluorobenzyl)-2-pyridinecarboxylate were treated in a manner similar to that described in Example 5, Step 1 to yield the product as an amber oil: ¹H NMR (CDCl₃) δ 7.89 (1H, d, J=1.4 Hz), 7.83 (1H, br t, J˜6 Hz), 7.15 (2H, dd, J˜9, 6 Hz), 6.98 (2H, t, J˜9 Hz), 6.92 (1H, s), 4.42 (2H, q, J=7 Hz), 3.92 (2H, s), 3.49 (2H, m), 3.41 (2H, t, J=7 Hz), 3.35 (2H, q, J=6 Hz), 2.36 (2H, t, J=8 Hz), 1.99 (2H, m), 1.42 (3H, t, J=7 Hz); HRMS calcd for C₂₁H₂₄FN₃O₃+H⁺: 386.1880. Found: 386.1880.

### Steps 3-4: Synthesis of ethyl 7-(4-fluorobenzyl)-4-hydroxy-2-oxo-1-[2-oxopyrrolidin-1-yl ethyl]-1,2-dihydro-1,5-naphthyridine-3-carboxylate

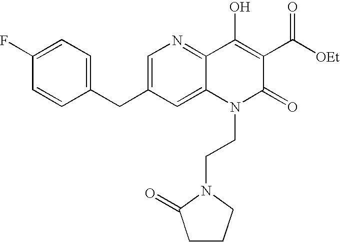

This compound was prepared from ethyl 5-(4-fluorobenzyl)-3-{[2-(2-oxopyrrolidin-1-yl)ethyl]amino}pyridine-2-carboxylate and ethyl 3-chloro-3-oxopropionate in a manner similar to that described in Example 1 and was obtained as a white solid: ¹H NMR (CDCl₃) δ 8.50 (1H, d, J=1.4 Hz), 8.11 (1H, s), 7.26 (2H, m), 7.00 (2H, ddd, J˜9, 9, 2 Hz), 4.52 (2H, q, J=7 Hz), 4.33 (2H, br t, J˜7 Hz), 4.14 (2H, s), 3.52-3.44 (4H, m), 2.35 (2H, t, J=8 Hz), 2.00 (2H, m), 1.48 (3H, t, J=7 Hz); HRMS calcd for C₂₄H₂₄FN₃O₅+H⁺: 454.1778. Found: 454.1787.

### EXAMPLE 18

### Diethyl {[({7-[(4-fluorophenyl)methyl]-4-hydroxy-2-oxo-1-[2-(2-oxo-1-pyrrolidinyl ethyl]-1,2-dihydro-1,5-naphthyridin-3-yl}carbonyl)amino]methyl}phosphonate

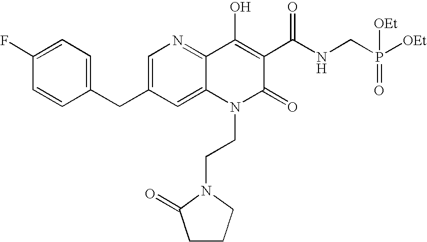

In a manner similar to that described in example 1, the title compound was prepared from_ethyl 7-(4-fluorobenzyl)-4-hydroxy-2-oxo-1-[2-(2-oxopyrrolidin-1-yl)ethyl]-1,2-dihydro-1,5-naphthyridine-3-carboxylate (85 mg, 0.19 mmol), diethyl (aminomethyl)phosphonate oxalate salt (145 mg, 0.56 mmol), and triethylamine (0.13 mL) as a white solid (37 mg, 34%) after purification by HPLC. ¹H NMR (CDCl₃) δ 10.44 (br, 1H), 8.55 (s, 1H), 8.04 (s, 1H), 7.22 (m, 2H), 6.98 (t, J=8.4 Hz, 3H), 4.34 (t, J=7.2 Hz, 2H), 4.21-4.13 (m, 4H), 4.13 (s, 2H), 3.89 (dd, J=12.8, 6.0 Hz, 2H), 3.49 (t, J=7.2 Hz, 2H), 3.42 (t, J=6.8 Hz, 2H), 2.30 (t, J=8.4 Hz, 2H), 1.97 (m, 2H), 1.33 (m, 6H); MS m/z 575 (M+1).

### EXAMPLE 19

### Ethyl hydrogen {[({7-[(4-fluorophenyl methyl]-4-hydroxy-2-oxo-1-[2-(2-oxo-1-pyrrolidinyl)ethyl]-1,2-dihydro-1,5-naphthyridin-3-yl}carbonyl)amino]methyl}phosphonate

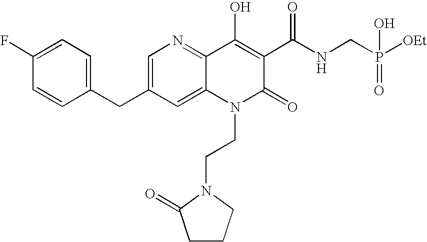

Diethyl {[({7-[(4-fluorophenyl)methyl]-4-hydroxy-2-oxo-1-[2-(2-oxo-1-pyrrolidinyl)ethyl]-1,2-dihydro-1,5-naphthyridin-3-yl}carbonyl)amino]methyl}phosphonate (10 mg, 0.017) was treated with sodium hydroxide in a manner similar to that described in example 3 to give the title compound (7 mg, 74%) as a white solid. ¹H NMR (CDCl₃) δ 10.41 (br s, 1H), 8.42 (s, 1H), 7.98 (s, 1H), 7.22 (m, 2H), 6.98 (m, 2H), 4.29 (m, 2H), 4.16 (m, 2H), 4.07 (s, 2H), 3.97-3.87 (m, 4H), 3.42 (m, 4H), 2.31 (m, 2H), 1.97 (m, 2H), 1.32 (m, 3H).

### EXAMPLE 20

### {[({7-[(4-Fluorophenyl)methyl]-4-hydroxy-2-oxo-1-[2-(2-oxo-1-pyrrolidinyl)ethyl]-1,2-dihydro-1,5-naphthyridin-3-yl}carbonyl)amino]methyl}phosphonic acid and 4-({2-[7-[(4-Fluorophenyl)methyl]4-hydroxy-2-oxo-3-{[(phosphonomethyl)amino]carbonyl}-1,5-naphthyridin-1(2H)-yl]ethyl}amino)butanoic acid.

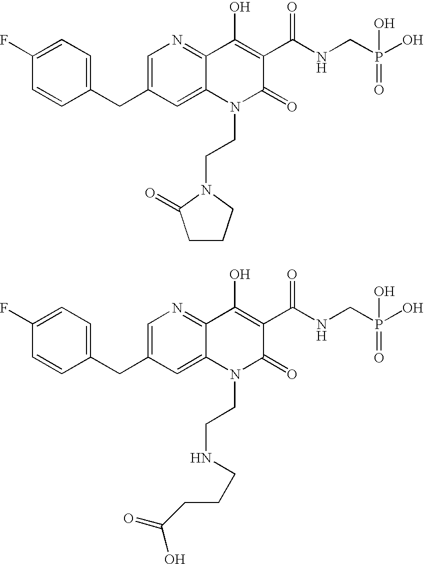 [0257] Diethyl {[({7-[(4-fluorophenyl)methyl]-4-hydroxy-2-oxo-1-[2-(2-oxo-1-pyrrolidinyl)ethyl]-1,2-dihydro-1,5-naphthyridin-3-yl}carbonyl)amino]methyl}phosphonate (11 mg, 0.019) was treated with 4 NHCl with microwave irradiation at 140° C. in a manner similar to that described in example 4 to give {[({7-[(4-Fluorophenyl)methyl]-4-hydroxy-2-oxo-1-[2-(2-oxo-1-pyrrolidinyl)ethyl]-1,2-dihydro-1,5-naphthyridin-3-yl}carbonyl)amino]methyl}phosphonic acid (3.5 mg, 34%) as a white solid and 4-({2-[7-[(4-fluorophenyl)methyl]-4-hydroxy-2-oxo-3-{[(phosphonomethyl)amino]carbonyl}-1,5-naphthyridin-1(2H)-yl]ethyl}amino)butanoic acid (2 mg, 20%) as a white solid which were purified by reverse phase preparative HPLC. For {[({7-[(4-Fluorophenyl)methyl]-4-hydroxy-2-oxo-1-[2-(2-oxo-1-pyrrolidinyl)ethyl]-1,2-dihydro-1,5-naphthyridin-3-yl}carbonyl)amino]methyl}phosphonic acid: ¹H NMR (CDCl₃/CD₃OD) δ 10.22 (br s, 1H), 8.28 (s, 1H), 7.76 (s, 1H), 7.05 (dd, J=8.4, 5.6 Hz, 2H), 6.81 (t, J=8.4 Hz, 2H), 4.04 (m, 1H), 3.97 (s, 2H), 3.57 (m, 2H), 3.32-3.25 (m, 4H), 2.10 (m, 2H), 1.79 (m, 2H); MS m/z 575 (M+1). For 4-({2-[7-[(4-fluorophenyl)methyl]-4-hydroxy-2-oxo-3-{[(phosphonomethyl)amino]carbonyl}-1,5-naphthyridin-1(2H)-yl]ethyl}amino)butanoic acid ¹H NMR (DMSO-d₆) δ 10.34 (br s, 1H), 8.49 (s, 1H), 8.11 (s, 1H), 7.37 (m, 2H), 7.11 (t, J=8.8 Hz, 2H), 4.54 (m, 2H), 4.12 (s, 2H), 3.09 (m, 2H), 2.98 (m, 2H), 2.30 (m, 2H), 1.79 (m, 2H), MS m/z 537 (M+1).

### EXAMPLE 21

### HIV Integrase Assay

Compounds were tested as inhibitors of recombinant HIV integrase in the following in vitro strand transfer assay. A complex of integrase and biotinylated donor DNA-streptavidin-coated SPA beads was formed by incubating 2 μM recombinant integrase with 0.66 μM biotinylated donor DNA-4 mg/ml streptavidin-coated SPA beads in 25 mM sodium MOPS pH 7.2, 23 mM NaCl, 10 mM MgCl₂, 10 mM dithiothreitol, and 10% DMSO for 5 minutes at 37° C. Beads were spun down, supernatant removed, and then beads resuspended in 25 mM sodium MOPS pH 7.2, 23 mM NaCl, 10 mM MgCl₂. Beads were again spun down, supernatant removed, and then beads resuspended in volume of 25 mM sodium MOPS pH 7.2, 23 mM NaCl, 10 mM MgCl₂ that would give 570 nM integrase (assuming all integrase bound the DNA-beads). Test compounds dissolved and diluted in DMSO were added to the integrase-DNA complex to give 6.7% DMSO (typically 1 μL of compound added to 14 μl of integrase complex), and preincubated for 60 minutes at 37° C. Then [3H] target DNA substrate was added to give a final concentration of 7 nM substrate, and the strand transfer reaction was incubated at 37° C. typically for 25 to 45 minutes which allowed for a linear increase in covalent attachment of the donor DNA to the radiolabelled target DNA. A 20 μl reaction was quenched by adding 60 μl of the following: 50 mM sodium EDTA pH 8, 25 mM sodium MOPS pH 7.2, 0.1 mg/ml salmon testes DNA, 500 mM NaCl. Streptavidin-coated SPA were from GE Healthcare, oligos to make the donor DNA were from Oligos Etc, and [3H] target DNA was a custom sythesis from Perkin Elmer. Sequences of donor and target DNA was described in Nucleic Acid Research 22, 1121-1122 (1994), with the addition of seven terminal A/T base pairs on each end of the target DNA that allowed for the incorporation of 14 tritiated T's (specific activity of target DNA approximately 1300 Ci/mmol). Compounds tested in this assay inhibited integrase with IC₅₀ values less than 100 nM.

What is claimed is:

1. A compound of formula (I): 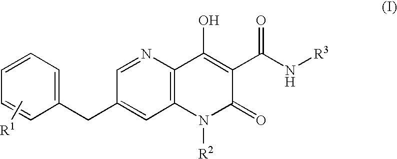
wherein:
R¹ is one or more substituents independently selected from hydrogen, hydroxy, CN, N(R^{a}R^{b}), C₁₋₈alkyl, C₃₋₇cycloalkyl, halogen and C₁₋₈alkoxy;
R² is selected from hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇cycloalkenyl, C₃₋₆alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘ N(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{a}), N(R^{a})C(SR^{a})═N(R^{b}) and heterocycle optionally substituted with oxo or R^{a};
or optionally when R² is C₅₋₇ cycloalkyl, C₆₋₁₄ aralkyl, C₅₋₇ cycloalkenyl, C₆₋₁₄ aryl or heterocycle R² may be fused to 5-7 membered carbocyclic or heterocyclic rings;
R³ is 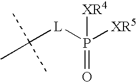
wherein:
L is C₁₋₈alkyl optionally substituted with C(O)NHR⁷;
X is O or NHR⁶;
R⁴ and R⁵ are independently hydrogen; C₁₋₈alkyl optionally substituted with C(O)R^{a} or C(O)NR^{a}R^{b}; or C₆₋₁₄aryl optionally substituted with halogen, alkoxy or NR^{a}R^{b};
R⁶ is hydrogen, C₁₋₈alkyl optionally substituted with OR⁷, or C₆₋₁₄aryl;
R⁷ is hydrogen or C₁₋₈alkyl;
R^{a} and R^{b} are independently hydrogen, NO₂, OR^{c}, CN, N(R^{c}R^{d}), C(O)R^{c}, C(O)C(O)R^{c}, C(O)N(R^{c}R^{d}), C(O)C(O)N(R^{c}R^{d}), S(O)ₘR^{c}, SR^{c}, S(O)ₘN(R^{c}R^{d}), C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄aryl, CN, NO₂, OR^{c}, N(R^{c}R^{d}), S(O)ₘR^{c}, SR^{c}, OS(O)ₘR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}), N(R^{c})S(O)ₘOR^{d}, C(O)R^{c}, OC(O)R^{c}, C(O)OR^{c}, OC(O)OR^{c}, N(R^{c}C)C(O)R^{d}, C(O)N(R^{c}R^{d}), N(R^{c})C(O)N(R^{c}R^{d}), OC(O)N(R^{c}R^{d}), N(R^{c}C)C(O)OR^{d}, C(NR^{c}R^{d})═N(R^{c}), C(SR^{c})═N(R^{d}), C(OR^{c})═N(R^{d}) and heterocycle;
Optionally, R^{a} and R^{b} may be linked together through one or more ring carbon atoms and/or ring heteroatoms including N, O, C(R^{c}R^{d}), C(O), S(O)ₘ, or S to form a saturated or unsaturated 3 to 8 membered carbocyclic or heterocyclic ring;
R^{c} and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;
Optionally, R^{c} and R^{d} may be linked together through one or more ring carbon atoms and/or ring heteroatoms including N, O, C(O) and S(O)ₘ, or S to form a saturated or unsaturated 3 to 8 membered carbocyclic or heterocyclic ring;
m is 1 or 2;
or a pharmaceutically acceptable salt thereof.

2. A compound of formula (I) according to claim 1 wherein:
R¹ is one or more substituents independently selected from hydrogen or halogen;
R² is
(a) hydrogen;
(b) C₁₋₈alkyl optionally substituted with C₃₋₇cycloalkyl, OR^{a}, N(R^{a}R^{b}), C(O)R^{a},
C(O)N(R^{a}R^{b}), or heterocycle optionally substituted with oxo or R^{a}; or
(c) C₆₋₁₄aryl optionally substituted with halogen;
R³ is 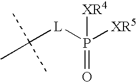
wherein
L is C₁₋₈alkyl optionally substituted with C(O)NHR⁷;
X is O or NHR⁶;
R⁴ and R⁵ are independently hydrogen; C₁₋₈alkyl optionally substituted with C(O)R^{a} or C(O)NR^{a}R^{b}; or C₆₋₁₄aryl optionally substituted with halogen, alkoxy or NR^{a}R^{b};
R⁶ is hydrogen, C₁₋₈alkyl optionally substituted with OR⁷, or C₆₋₁₄aryl;
R⁷ is hydrogen or C₁₋₈alkyl;
wherein R^{a} and R^{b} are independently hydrogen, OR^{c}, SR^{c}, C₁₋₈alkyl, C₆₋₁₄aryl or heterocycle, each of which each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, CN, NO₂, OR^{c}, N(R^{c}R^{d}), S(O)ₘR^{c}, SR^{c}, OS(O)ₘR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}), N(R^{c})S(O)ₘOR^{d}, C(O)R^{c}, OC(O)R^{c}, C(O)OR^{c}, OC(O)OR^{c}, N(R^{c}C)C(O)R^{d}, C(O)N(R^{c}R^{d}), N(R^{c})C(O)N(R^{c}R^{d}), OC(O)N(R^{c}R^{d}), N(R^{c}C)C(O)OR^{d}, C(NR^{c}R^{d})═N(R^{c}), C(SR^{c})═N(R^{d}), C(OR^{c})═N(R^{d}) and heterocycle;
wherein R^{c} is hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;
R^{c} and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;
or a pharmaceutically acceptable salt thereof.

3. A compound of formula (I) according to claim 1 wherein:
R¹ is one or more substituents independently selected from hydrogen or halogen;
R² is
(a) hydrogen;
(b) C₁₋₈alkyl optionally substituted with C₃₋₇cycloalkyl, OR^{a}, N(R^{a}R^{b}), C(O)R^{a}, C(O)N(R^{a}R^{b}), or heterocycle optionally substituted with oxo or R^{a}; or
(c) C₆₋₁₄aryl optionally substituted with halogen;
R³ is 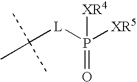
wherein
L is C₁₋₈alkyl optionally substituted with C(O)NHR⁷;
X is O or NHR⁶;
R⁴ and R⁵ are independently hydrogen; C₁₋₈alkyl optionally substituted with C(O)R^{a} or C(O)NR^{a}R^{b}; or C₆₋₁₄aryl optionally substituted with halogen, alkoxy or NR^{a}R^{b};
R⁶ is hydrogen, C₁₋₈alkyl optionally substituted with OR⁷, or C₆₋₁₄aryl;
R⁷ is hydrogen or C₁₋₈alkyl;
wherein R^{a} and R^{b} are independently hydrogen, NO₂, OR^{c}, C(O)R^{c}, C₁₋₈alkyl optionally substituted with OR^{c}, C(O)OR^{c}, C₆₋₁₄aryl or heterocycle;
wherein R^{c} is hydrogen, C₁₋₈ alkyl or C₆₋₁₄aryl;
or a pharmaceutically acceptable salt thereof.

4. A compound of formula (I) according to claim 1 wherein:
R¹ is one or more substituents independently selected from hydrogen or halogen;
R² is C₁₋₈alkyl optionally substituted with C₃₋₇cycloalkyl, OR^{a}, N(R^{a}R^{b}), C(O)R^{a}, C(O)N(R^{a}R^{b}), or heterocycle optionally substituted with oxo; or C₆₋₁₄aryl optionally substituted with halogen;
R³ is 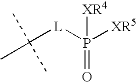
wherein
L is absent or C₆₋₁₄aryl or C₁₋₈alkyl optionally substituted with C(O)NHR⁷;
X is O or NHR⁶;
R⁴ and R⁵ are independently hydrogen; C₁₋₈alkyl optionally substituted with C(O)R^{a} or C(O)NR^{a}R^{b}; or C₆₋₁₄aryl optionally substituted with halogen, alkoxy or NR^{a}R^{b};
R⁶ is hydrogen, C₁₋₈alkyl optionally substituted with OR⁷, or C₆₋₁₄aryl;
R⁷ is hydrogen or C₁₋₈alkyl;
wherein R^{a} and R^{b} are independently hydrogen, NO₂, OR^{c}, C(O)R^{c}, C₁₋₈alkyl optionally substituted with OR^{c}, C(O)OR^{c}, C₆₋₁₄aryl or heterocycle;
wherein R^{c} is hydrogen, C₁₋₈ alkyl or C₆₋₁₄aryl;
or a pharmaceutically acceptable salt thereof.

5. A compound of formula (I) according to claim 1 wherein L is C₁₋₈ alkyl or a pharmaceutically acceptable salt thereof.

6. A compound of formula (I) according to claim 1 wherein L is C₁₋₈ alkyl and X is O or a pharmaceutically acceptable salt thereof.

7. A compound selected from the group consisting of:
Diethyl{2-[({7-[(4-fluorophenyl)methyl]-4-hydroxy-2-oxo-1,2-dihydro-1,5-naphthyridin-3-yl}carbonyl)amino]ethyl}phosphonate;
Diethyl{2-[({7-[(4-fluorophenyl)methyl]-4-hydroxy-1-methyl-2-oxo-1,2-dihydro-1,5-naphthyridin-3-yl}carbonyl)amino]ethyl}phosphonate;
Ethyl hydrogen {2-[({7-[(4-fluorophenyl)methyl]-4-hydroxy-1-methyl-2-oxo-1,2-dihydro-1,5-naphthyridin-3-yl}carbonyl)amino]ethyl}phosphonate;
{2-[({7-[(4-Fluorophenyl)methyl]-4-hydroxy-1-methyl-2-oxo-1,2-dihydro-1,5-naphthyridin-3-yl}carbonyl)amino]ethyl}phosphonic acid;
Ethyl P-{2-[({7-[(4-fluorophenyl)methyl]-4-hydroxy-1-methyl-2-oxo-1,2-dihydro-1,5-naphthyridin-3-yl}carbonyl)amino]ethyl}-N-[2-(methyloxy)ethyl]phosphonamidate;
Diethyl{4-[({7-[(4-fluorophenyl)methyl]-4-hydroxy-1-methyl-2-oxo-1,2-dihydro-1,5-naphthyridin-3-yl}carbonyl)amino]butyl}phosphonate;
Ethyl hydrogen {4-[({7-[(4-fluorophenyl)methyl]-4-hydroxy-1-methyl-2-oxo-1,2-dihydro-1,5-naphthyridin-3-yl}carbonyl)amino]butyl}phosphonate;
{4-[({7-[(4-Fluorophenyl)methyl]-4-hydroxy-1-methyl-2-oxo-1,2-dihydro-1,5-naphthyridin-3-yl}carbonyl)amino]butyl}phosphonic acid;
Diethyl{3-[({7-[(4-fluorophenyl)methyl]-4-hydroxy-1-methyl-2-oxo-1,2-dihydro-1,5-naphthyridin-3-yl}carbonyl)amino]propyl}phosphonate;
Ethyl hydrogen {3-[({7-[(4-fluorophenyl)methyl]-4-hydroxy-1-methyl-2-oxo-1,2-dihydro-1,5-naphthyridin-3-yl}carbonyl)amino]propyl}phosphonate;
{3-[({7-[(4-Fluorophenyl)methyl]-4-hydroxy-1-methyl-2-oxo-1,2-dihydro-1,5-naphthyridin-3-yl}carbonyl)amino]propyl}phosphonic acid;
Diethyl {[({7-[(4-fluorophenyl)methyl]-4-hydroxy-1-methyl-2-oxo-1,2-dihydro-1,5-naphthyridin-3-yl}carbonyl)amino]methyl}phosphonate;
Ethyl hydrogen {[({7-[(4-fluorophenyl)methyl]-4-hydroxy-1-methyl-2-oxo-1,2-dihydro-1,5-naphthyridin-3-yl}carbonyl)amino]methyl}phosphonate;
{[({7-[(4-Fluorophenyl)methyl]-4-hydroxy-1-methyl-2-oxo-1,2-dihydro-1,5-naphthyridin-3-yl}carbonyl)amino]methyl}phosphonic acid;
Diethyl {2-[({1-[2-(dimethylamino)-2-oxoethyl]-7-[(4-fluorophenyl)methyl]-4-hydroxy-2-oxo-1,2-dihydro-1,5-naphthyridin-3-yl}carbonyl)amino]ethyl}phosphonate;
Diethyl {[({1-[2-(dimethylamino)-2-oxoethyl]-7-[(4-fluorophenyl)methyl]-4-hydroxy-2-oxo-1,2-dihydro-1,5-naphthyridin-3-yl}carbonyl)amino]methyl}phosphonate;
Diethyl{2-[({1-(4-fluorophenyl)-7-[(4-fluorophenyl)methyl]-4-hydroxy-2-oxo-1,2-dihydro-1,5-naphthyridin-3-yl}carbonyl)amino]ethyl}phosphonate;
Diethyl{[({7-[(4-fluorophenyl)methyl]-4-hydroxy-2-oxo-1-[2-(2-oxo-1-pyrrolidinyl)ethyl]-1,2-dihydro-1,5-naphthyridin-3-yl}carbonyl)amino]methyl}phosphonate;
Ethyl hydrogen {[({7-[(4-fluorophenyl)methyl]-4-hydroxy-2-oxo-1-[2-(2-oxo-1-pyrrolidinyl)ethyl]-1,2-dihydro-1,5-naphthyridin-3-yl}carbonyl)amino]methyl}phosphonate;
{[({7-[(4-Fluorophenyl)methyl]-4-hydroxy-2-oxo-1-[2-(2-oxo-1-pyrrolidinyl)ethyl]-1,2-dihydro-1,5-naphthyridin-3-yl}carbonyl)amino]methyl}phosphonic acid;
4-({2-[7-[(4-Fluorophenyl)methyl]-4-hydroxy-2-oxo-3-{[(phosphonomethyl)amino]carbonyl}-1,5-naphthyridin-1(2H)-yl]ethyl}amino)butanoic acid;
and pharmaceutically acceptable salts thereof.

8. A method of treatment of a viral infection in a human comprising administering to said human an antiviral effective amount of a compound according to claim 1.

9. A method according to claim 8 wherein the viral infection is a HIV infection.

10. (canceled)

11. (canceled)

12. (canceled)

13. A pharmaceutical composition comprising an effective amount of a compound according to claim 1 together with a pharmaceutically acceptable carrier.

14. A pharmaceutical composition according to claim 13 in the form of a tablet or capsule.

15. A pharmaceutical composition according to claim 13 in the form of a liquid or suspension.

16. A method of treatment of a viral infection in a human comprising administering to said human a composition comprising a compound according to claim 1 and another therapeutic agent.

17. The method according to claim 16 wherein the viral infection is an HIV infection.

18. A composition according to claim 13, wherein said composition comprises at least one additional therapeutic agent selected from the group consisting of (1-alpha, 2-beta, 3-alpha)-9-[2,3-bis(hydroxymethyl)cyclobutyl]guanine [(−)BHCG, SQ-34514, lobucavir], 9-[(2R,3R,4S)-3,4-bis(hydroxymethyl)-2-oxetanosyl]adenine (oxetanocin-G), TMC-114, BMS-232632, acyclic nucleosides [e.g. acyclovir, valaciclovir, famciclovir, ganciclovir, penciclovir), acyclic nucleoside phosphonates [e.g. (S)-1-(3-hydroxy-2-phosphonyl-methoxypropyl)cytosine (HPMPC), [[[2-(6-amino-9H-purin-9-yl)ethoxy]methyl]phosphinylidene]bis(oxymethylene)-2,2-dimethylpropanoic acid (bis-POM PMEA, adefovir dipivoxil), [[(1R)-2-(6-amino-9H-purin-9-yl)-1-methylethoxy]methyl]phosphonic acid (tenofovir), (R)-[[2-(6-Amino-9H-purin-9-yl)-1-methylethoxy]methyl]phosphonic acid bis-(isopropoxycarbonyloxymethyl)ester (bis-POC-PMPA)], ribonucleotide reductase inhibitors (e.g. 2-acetylpyridine 5-[(2-chloroanilino)thiocarbonyl) thiocarbonohydrazone and hydroxyurea), nucleoside reverse transcriptase inhibitors (e.g., 3′-azido-3′-deoxythymidine (AZT, zidovudine), 2′,3′-dideoxycytidine (ddC, zalcitabine), 2′,3′-dideoxyadenosine, 2′,3′-dideoxyinosine (ddI, didanosine), 2′,3′-didehydrothymidine (d4T, stavudine), (−)-beta-D-2,6-diaminopurine dioxolane (DAPD), 3′-Azido-2′,3′-dideoxythymidine-5′-H-phosphosphonate (phosphonovir), 2′-deoxy-5-iodo-uridine (idoxuridine), as (−)-cis-1-(2-hydroxymethyl)-1,3-oxathiolane 5-yl)-cytosine (lamivudine), or cis-1-(2-(hydroxymethyl)-1,3-oxathiolan-5-yl)-5-fluorocytosine (FTC), 3′-deoxy-3′-fluorothymidine, 5-chloro-2′,3′-dideoxy-3′-fluorouridine, (−)-cis-4-[2-amino-6-(cyclopropylamino)-9H-purin-9-yl]-2-cyclopentene-1-methanol (abacavir), 9-[4-hydroxy-2-(hydroxymethyl)but-1-yl]-guanine (H2G), ABT-606 (2HM-H2G) and ribavirin), protease inhibitors (e.g. indinavir, ritonavir, nelfinavir, amprenavir, saquinavir, (R)—N-tert-butyl-3-[(2S,3S)-2-hydroxy-3-N-[(R)-2-N-(isoquinolin-5-yloxyacetyl)amino-3-methylthiopropanoyl]amino-4-phenylbutanoyl]-5,5-dimethyl-1,3-thiazolidine-4-carboxamide (KNI-272), 4R-(4-alpha,5alpha,6beta)]-1,3-bis[(3-aminophenyl)methyl]hexahydro-5,6-dihydroxy-4,7-bis(phenylmethyl)-2H-1,3-diazepin-2-one dimethanesulfonate (mozenavir), 3-[1-[3-[2-(5-trifluoromethylpyridinyl)-sulfonylamino]phenyl]propyl]-4-hydroxy-6alpha-phenethyl-6beta-propyl-5,6-dihydro-2-pyranone (tipranavir), N′-[2(S)-Hydroxy-3(S)—[N-(methoxycarbonyl)-1-tert-leucylamino]-4-phenylbutyl-N^{alpha}-(methoxycarbonyl)-N′-[4-(2-pyridyl)benzyl]-L-tert-leucylhydrazide (BMS-232632), 3-(2(S)-Hydroxy-3(S)-(3-hydroxy-2-methylbenzamido)-4-phenylbutanoyl)-5,5-dimethyl-N-(2-methylbenzyl)thiazolidine-4(R)-carboxamide (AG-1776), N-(2(R)-Hydroxy-1(S)-indanyl)-2(R)-phenyl-methyl-4(S)-hydroxy-5-(1-(1-(4-benzo[b]furanylmethyl)-2(S)—N′-(tert-butylcarboxamido)piperazinyl)pentanamide (MK-944A), and GW 433908), interferons such as α-interferon, renal excretion inhibitors such as probenecid, nucleoside transport inhibitors such as dipyridamole; pentoxifylline, N-acetylcysteine (NAC), Procysteine, α-trichosanthin, phosphonoformic acid, as well as immunomodulators such as interleukin II or thymosin, granulocyte macrophage colony stimulating factors, erythropoetin, soluble CD₄ and genetically engineered derivatives thereof, non-nucleoside reverse transcriptase inhibitors (NNRTIs) for example, TMC-120, TMC-125, nevirapine (BI-RG-587), alpha-((2-acetyl-5-methylphenyl)amino)-2,6-dichloro-benzeneacetamide (loviride), 1-[3-(isopropylamino)-2-pyridyl]-4-[5-(methanesulfonamido)-1H-indol-2-ylcarbonyl]piperazine monomethanesulfonate (delavirdine), (10R,11S, 12S)-12-Hydroxy-6,6,10,11-tetramethyl-4-propyl-11,12-dihydro-2H, 6H, 10H-benzo(1,2-b:3, 4-b′:5,6-b″)tripyran-2-one ((+) calanolide A), (4S)-6-Chloro-4-[1E)-cyclopropylethenyl)-3,4-dihydro-4-(trifluoromethyl)-2(1H)-quinazolinone (DPC-083), 1-(ethoxymethyl)-5-(1-methylethyl)-6-(phenylmethyl)-2,4(1H,3H)-pyrimidinedione (MKC-442), 5-(3,5-dichlorophenyl)thio-4-isopropyl-1-(4-pyridyl)methyl-1H-imidazol-2-ylmethyl carbamate (capravirine), glycoprotein 120 antagonists [e.g. PRO-2000, PRO-542 and 1,4-bis[3-[(2,4-dichlorophenyl)carbonylamino]-2-oxo-5,8-disodiumsulfanyl]naphthalyl-2,5-dimethoxyphenyl-1,4-dihydrazone (FP-21399)], cytokine antagonists [e.g. reticulose (Product-R), 1,1′-azobis-formamide (ADA), and 1,11-(1,4-phenylenebis(methylene))bis-1,4,8,11-tetraazacyclotetradecane octahydrochloride (AMD-3100)], and fusion inhibitors for example T-20 and T-124.

19. A method according to claim 16, wherein said therapeutic agent is selected from the group consisting of (1-alpha, 2-beta, 3-alpha)-9-[2,3-bis(hydroxymethyl)cyclobutyl]guanine [(−)BHCG, SQ-34514, lobucavir], 9-[(2R,3R,4S)-3,4-bis(hydroxymethyl)-2-oxetanosyl]adenine (oxetanocin-G), acyclic nucleosides [e.g. acyclovir, valaciclovir, famciclovir, ganciclovir, penciclovir), acyclic nucleoside phosphonates [e.g. (S)-1-(3-hydroxy-2-phosphonyl-methoxypropyl)cytosine (HPMPC), [[[2-(6-amino-9H-purin-9-yl)ethoxy]methyl]phosphinylidene]bis(oxymethylene)-2,2-dimethylpropanoic acid (bis-POM PMEA, adefovir dipivoxil), [[(1R)-2-(6-amino-9H-purin-9-yl)-1-methylethoxy]methyl]phosphonic acid (tenofovir), (R)-[[2-(6-Amino-9H-purin-9-yl)-1-methylethoxy]methyl]phosphonic acid bis-(isopropoxycarbonyloxymethyl)ester (bis-POC-PMPA)], ribonucleotide reductase inhibitors (e.g. 2-acetylpyridine 5-[(2-chloroanilino)thiocarbonyl) thiocarbonohydrazone and hydroxyurea), nucleoside reverse transcriptase inhibitors (e.g., 3′-azido-3′-deoxythymidine (AZT, zidovudine), 2′,3′-dideoxycytidine (ddC, zalcitabine), 2′,3′-dideoxyadenosine, 2′,3′-dideoxyinosine (ddI, didanosine), 2′,3′-didehydrothymidine (d4T, stavudine), (−)-beta-D-2,6-diaminopurine dioxolane (DAPD), 3′-Azido-2′,3′-dideoxythymidine-5′-H-phosphosphonate (phosphonovir), 2′-deoxy-5-iodo-uridine (idoxuridine), as (−)-cis-1-(2-hydroxymethyl)-1,3-oxathiolane 5-yl)-cytosine (lamivudine), or cis-1-(2-(hydroxymethyl)-1,3-oxathiolan-5-yl)-5-fluorocytosine (FTC), 3′-deoxy-3′-fluorothymidine, 5-chloro-2′,3′-dideoxy-3′-fluorouridine, (−)-cis-4-[2-amino-6-(cyclopropylamino)-9H-purin-9-yl]-2-cyclopentene-1-methanol (abacavir), 9-[4-hydroxy-2-(hydroxymethyl)but-1-yl]-guanine (H2G), ABT-606 (2HM-H2G) and ribavirin), protease inhibitors (e.g. indinavir, ritonavir, nelfinavir, amprenavir, saquinavir, (R)—N-tert-butyl-3-[(2S,3S)-2-hydroxy-3-N—[(R)-2-N-(isoquinolin-5-yloxyacetyl)amino-3-methylthiopropanoyl]amino-4-phenylbutanoyl]-5,5-dimethyl-1,3-thiazolidine-4-carboxamide (KNI-272), 4R-(4-alpha,5alpha,6beta)]-1,3-bis[(3-aminophenyl)methyl]hexahydro-5,6-dihydroxy-4,7-bis(phenylmethyl)-2H-1,3-diazepin-2-one dimethanesulfonate (mozenavir), 3-[1-[3-[2-(5-trifluoromethylpyridinyl)-sulfonylamino]phenyl]propyl]-4-hydroxy-6alpha-phenethyl-6beta-propyl-5,6-dihydro-2-pyranone (tipranavir), N′-[2(S)-Hydroxy-3(S)—[N-(methoxycarbonyl)-1-tert-leucylamino]-4-phenylbutyl-N^{alpha}-(methoxycarbonyl)-N′-[4-(2-pyridyl)benzyl]-L-tert-leucylhydrazide (BMS-232632), 3-(2(S)-Hydroxy-3(S)-(3-hydroxy-2-methylbenzamido)-4-phenylbutanoyl)-5,5-dimethyl-N-(2-methylbenzyl)thiazolidine-4(R)-carboxamide (AG-1776), N-(2(R)-Hydroxy-1(S)-indanyl)-2(R)-phenyl-methyl-4(S)-hydroxy-5-(1-(1-(4-benzo[b]furanylmethyl)-2(S)—N′-(tert-butylcarboxamido)piperazinyl)pentanamide (MK-944A), and GW 433908), interferons such as α-interferon, renal excretion inhibitors such as probenecid, nucleoside transport inhibitors such as dipyridamole; pentoxifylline, N-acetylcysteine (NAC), Procysteine, α-trichosanthin, phosphonoformic acid, as well as immunomodulators such as interleukin II or thymosin, granulocyte macrophage colony stimulating factors, erythropoetin, soluble CD₄ and genetically engineered derivatives thereof, non-nucleoside reverse transcriptase inhibitors (NNRTIs) [e.g. nevirapine (BI-RG-587), alpha-((2-acetyl-5-methylphenyl)amino)-2,6-dichloro-benzeneacetamide (loviride), 1-[3-(isopropylamino)-2-pyridyl]-4-[5-(methanesulfonamido)-1H-indol-2-ylcarbonyl]piperazine monomethanesulfonate (delavirdine), (10R, 11S, 12S)-12-Hydroxy-6,6,10,11-tetramethyl-4-propyl-11,12-dihydro-2H, 6H, 10H-benzo(1, 2-b:3, 4-b′:5, 6-b″)tripyran-2-one ((+) calanolide A), (4S)-6-Chloro-4-[1E)-cyclopropylethenyl)-3,4-dihydro-4-(trifluoromethyl)-2(1H)-quinazolinone (DPC-083), 1-(ethoxymethyl)-5-(1-methylethyl)-6-(phenylmethyl)-2,4(1H,3H)-pyrimidinedione (MKC-442), 5-(3,5-dichlorophenyl)thio-4-isopropyl-1-(4-pyridyl)methyl-1H-imidazol-2-ylmethyl carbamate (capravirine)], glycoprotein 120 antagonists [e.g. PRO-2000, PRO-542 and 1,4-bis[3-[(2,4-dichlorophenyl)carbonylamino]-2-oxo-5,8-disodiumsulfanyl]naphthalyl-2,5-dimethoxyphenyl-1,4-dihydrazone (FP-21399)], cytokine antagonists [e.g. reticulose (Product-R), 1,1′-azobis-formamide (ADA), and 1,11-(1,4-phenylenebis(methylene))bis-1,4,8,11-tetraazacyclotetradecane octahydrochloride (AMD-3100)], and fusion inhibitors (e.g. T-20 and T-1249).
